(19) United States
(12) Patent Application Publication
(10) Pub. No.: US 2017/0325494 A1
(43) Pub. Date: Nov. 16, 2017

(54) LIQUID COMPOSITION CONTAINING NICOTINE FROM NON-TOBACCO SOURCE FOR USE WITH ELECTRONIC VAPORIZING DEVICES

(71) Applicant: LunaTech, LLC, Encino, CA (US)

(72) Inventors: John David Cameron, Encino, CA (US); Dean Becker, Fairhope, AL (US); Gene Fein, Oxnard, CA (US)

(21) Appl. No.: 15/596,373

(22) Filed: May 16, 2017

Related U.S. Application Data
(63) Continuation-in-part of application No. 15/493,804, filed on Apr. 21, 2017.
(60) Provisional application No. 62/337,067, filed on May 16, 2016.

(51) Int. Cl. A24B 15/16; A61K 9/00

(52) U.S. Cl. CPC A24B 15/167; A61K 9/007

(57) ABSTRACT

The present disclosure is directed to a liquid composition containing nicotine derived from non-tobacco nicotine sources for use with electronic vaporizing devices. The present disclosure is also directed to electronic vaporizing devices for using such composition. In one embodiment, the liquid compositions are water-based and are substantially free of propylene glycol and vegetable glycerin. In a preferred embodiment, the compositions may be augmented to include additives such as flavorings, medicines, wellness elements, recreational elements, and emulsifiers.

### CROSS-REFERENCE TO RELATED APPLICATION

This application claims the benefit of U.S. Provisional Application No. 62/337,067, filed on May 16, 2016, entitled “Composition Extracted from Duboisia Hopwoodii and Methods of Using Same”, and U.S. Non-Provisional patent application Ser. No. 15/493,804, filed on Apr. 21, 2017, entitled “Natural-Based Liquid Compositions and Electronic Vaporizing Devices for Using Such Compositions”, the contents of which are incorporated herein by reference as though set forth in their entireties.

### BACKGROUND

The present disclosure is directed to a liquid composition containing nicotine derived from non-tobacco nicotine sources for use with electronic vaporizing devices. The present disclosure is also directed to electronic vaporizing devices for using such composition. In one embodiment, the liquid compositions are water-based and are substantially free of propylene glycol and vegetable glycerin. In a preferred embodiment, the compositions may be augmented to include additives such as flavorings, medicines, wellness elements, recreational elements, and emulsifiers.

Consumers utilize electronic vapor cigarettes, pipes, and modified vapor devices to enjoy what is commonly known as “vaping.” Vaping is an increasingly popular market segment, which has been steadily gaining market share over the last several years, and continues to do so. In general, currently available vaporizers are characterized by heating a solid to a smoldering point, vaporizing a liquid by direct or indirect heat, or nebulizing a liquid by heat and/or by expansion through a nozzle.

The majority of commercially used nicotine is obtained from the tobacco plant. Unfortunately, the tobacco plant also contains many components that are detrimental to the consumer including, for example, known carcinogens (e.g., acrolein) and respiratory toxicants (e.g., acrolein, methyl ethyl ketone).

Therefore, it would be desirable to provide a liquid composition for electronic vaporizing devices that contains nicotine from non-tobacco sources to improve the vaping experience, long-term safety, and lifestyle.

### SUMMARY

The following presents a simplified overview of the example embodiments in order to provide a basic understanding of some embodiments of the example embodiments. This overview is not an extensive overview of the example embodiments. It is intended to neither identify key or critical elements of the example embodiments nor delineate the scope of the appended claims. Its sole purpose is to present some concepts of the example embodiments in a simplified form as a prelude to the more detailed description that is presented hereinbelow. It is to be understood that both the following general description and the following detailed description are exemplary and explanatory only and are not restrictive.

In accordance with the embodiments disclosed herein, the present disclosure is directed to a vaporizable liquid composition, methods for producing the vaporizable liquid composition, and methods for using the vaporizable liquid composition. In one embodiment, a liquid composition may be provided for use with electronic vaporizing devices. In one embodiment, a liquid composition may be provided for use in an electronic vaporizing device having a cold-vapor system wherein the vapor is not heated, but dispersed into a mist.

In one embodiment, a liquid composition may be provided that comprises a carrier and nicotine derived from at least one non-tobacco nicotine source. In a preferred embodiment, a liquid composition may be provided comprising nicotine and nornicotine.

In one embodiment, a method may be provided for producing a liquid composition for use in electronic vaporizing devices, wherein the liquid composition comprises nicotine derived from at least one non-tobacco source. In another embodiment, a method may be provided for extracting nicotine from the at least one non-tobacco source for use in the liquid composition.

In accordance with the embodiments disclosed herein, the present disclosure may provide a natural-based liquid composition for use in an electronic vaporizing device. The natural-based liquid composition may comprise about 70 weight percent to about 99 weight percent of water and about 0.01 weight percent to about 10 weight percent of nicotine derived from at least one non-tobacco nicotine source, wherein the natural-based liquid composition may be substantially free of at least one of propylene glycol and vegetable glycerin. In one embodiment, the natural-based liquid composition may be substantially free of both propylene glycol and vegetable glycerin.

In one embodiment, the at least one non-tobacco nicotine source may be selected from the Solanaceae plant family. In a preferred embodiment, the at least one non-tobacco nicotine source may be selected from the group of non-tobacco nicotine sources consisting of at least one of duboisia hopwoodii, eggplant, tomatoes, potatoes, green peppers, coca, and combinations thereof. In a more preferred embodiment, the at least one non-tobacco nicotine source may be duboisia hopwoodii. In one embodiment, the natural-based liquid composition may further comprise about 0.01 weight percent to about 10 weight percent of nornicotine derived from at least one non-tobacco nornicotine source.

In one embodiment, the natural-based liquid composition may further comprise about 1 weight percent to about 30 weight percent of at least one flavoring agent. In another embodiment, the at least one flavoring agent may be selected from the group of flavoring agents consisting of at least one of a fruit, a berry, a spice, an herb, a savory flavor, a spicy flavor, a sweet flavor, a plant-based flavor, and combinations thereof.

In one embodiment, the natural-based liquid composition may further comprise about 0.01 weight percent to about 20 weight percent of at least one natural emulsifier. In another embodiment, the at least one natural emulsifier may be selected from the group of natural emulsifiers consisting of at least one of a sunflower, a lecithin, a natural starch, and combinations thereof.

In another embodiment, the natural-based liquid composition may further comprise about 0.01 weight percent to about 10 weight percent of at least one supplementary component selected from the group of supplementary components consisting of at least one of a medicinal agent, a wellness agent, a recreational use agent, and combinations thereof. In one embodiment, the supplementary component may be a medicinal agent that is selected from the group of medicinal agents consisting of at least one of a diabetes medication, a respiratory medication, a sexual dysfunction medication, a cannabis-based medication, and combinations thereof. In another embodiment, the supplementary component may be a wellness agent that is selected from the group wellness agents consisting of at least one of a chamomile, Echinacea, a homeopathic remedy, a vitamin supplement, and combinations thereof. In another embodiment, the supplementary component may be a recreational use agent that is selected from the group of recreational use agents consisting of at least one of caffeine, a cannabis-based material, taurine, salvia, kratum, kava, and combinations thereof.

In accordance with the embodiments disclosed herein, the present disclosure may provide a natural-based liquid composition for use in an electronic vaporizing device. The natural-based liquid may comprise about 70 weight percent to about 99 weight percent of water; about 0.01 weight percent to about 10 weight percent of nicotine derived from at least one non-tobacco nicotine source; and about 0.01 weight percent to about 20 weight percent of at least one natural emulsifier; wherein the natural-based liquid composition is substantially free of both propylene glycol and vegetable glycerin.

In accordance with the embodiments disclosed herein, the present disclosure may provide a method for producing a natural-based liquid composition for use in an electronic vaporizing device. The method may comprise the steps of combining about 70 weight percent to about 99 weight percent of water with about 0.01 weight percent to about 10 weight percent of nicotine derived from at least one non-tobacco nicotine source to form a liquid solution; adding about 0.01 weight percent to about 20 weight percent of at least one natural emulsifier to the liquid solution; and mixing the liquid composition containing the at least one natural emulsifier to form a substantially homogeneous liquid composition.

In one embodiment, the at least one non-tobacco nicotine source may be selected from the Solanaceae plant family. In a preferred embodiment, the at least one non-tobacco nicotine source may be selected from the group of non-tobacco nicotine sources consisting of at least one of duboisia hopwoodii, eggplant, tomatoes, potatoes, green peppers, coca, and combinations thereof. In one embodiment, the at least one natural emulsifier may be selected from the group of natural emulsifiers consisting of at least one of a sunflower, a lecithin, a natural starch, and combinations thereof.

In one embodiment, the method may further comprise adding about 0.01 weight percent to about 10 weight percent of at least one supplementary component to the liquid solution, wherein the at least one supplementary component is selected from the group of supplementary components consisting of at least one of a flavoring agent, a medicinal agent, a wellness agent, a recreational use agent, nornicotine, and combinations thereof.

Still other advantages, embodiments, and features of the subject disclosure will become readily apparent to those of ordinary skill in the art from the following description wherein there is shown and described a preferred embodiment of the present disclosure, simply by way of illustration of one of the best modes best suited to carry out the subject disclosure. As it will be realized, the present disclosure is capable of other different embodiments and its several details are capable of modifications in various obvious embodiments all without departing from, or limiting, the scope herein. Accordingly, the drawings and descriptions will be regarded as illustrative in nature and not as restrictive.

### BRIEF DESCRIPTION OF THE DRAWINGS

The drawings are of illustrative embodiments. They do not illustrate all embodiments. Other embodiments may be used in addition or instead. Details which may be apparent or unnecessary may be omitted to save space or for more effective illustration. Some embodiments may be practiced with additional components or steps and/or without all of the components or steps which are illustrated. When the same numeral appears in different drawings, it refers to the same or like components or steps.

FIG. 1 illustrates a block diagram of one embodiment of an electronic vaporizing device according to some embodiments.

FIG. 2 is an illustration of one embodiment of an electronic vaporizing device according to some embodiments.

FIG. 3 is an illustration of one embodiment of an electronic vaporizing device configured for vaporizing a mixture of vaporizable material according to some embodiments.

FIG. 4 is an illustration of one embodiment of an electronic vaporizing device configured for smooth vapor delivery according to some embodiments.

FIG. 5 is an illustration of one embodiment of an electronic vaporizing device configured for smooth vapor delivery according to some embodiments.

FIG. 6 is an illustration of one embodiment of an electronic vaporizing device configured for smooth vapor delivery according to some embodiments.

FIG. 7 is an illustration of one embodiment of an electronic vaporizing device configured for smooth vapor delivery according to some embodiments.

FIG. 8 is an illustration of one embodiment of an electronic vaporizing device configured for filtering air according to some embodiments.

FIG. 9 illustrates one embodiment of an interface for an electronic vaporizing device according to some embodiments.

FIG. 10 illustrates one embodiment of an interface for an electronic vaporizing device according to some embodiments.

FIG. 11 is a flow block diagram of one embodiment of a method for producing a liquid composition for use in an electronic vaporizing device according to some embodiments.

### DETAILED DESCRIPTION OF THE ILLUSTRATIVE EMBODIMENTS

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

As used herein, the term “by weight,” when used in conjunction with a component, unless specially stated to the contrary is based on the total weight of the formulation or composition in which the component is included. For example, if a particular element or component in a composition or article is said to have 8% by weight, it is understood that this percentage is in relation to a total compositional percentage of 100%.

A weight percent of a component, or weight %, or weight percent, or weight % (percent) unless specifically stated to the contrary, is based on the total weight of the formulation or composition in which the component is included.

References in the specification and concluding claims to parts by weight of a particular element or component in a composition or article, denotes the weight relationship between the element or component and any other elements or components in the composition or article for which a part by weight is expressed. Thus, in a composition or a selected portion of a composition containing 2 parts by weight of component X and 5 parts by weight component Y, X and Y are present at a weight ratio of 2:5, and are present in such ratio regardless of whether additional components are contained in the composition.

In the following description, certain terminology is used to describe certain features of one or more embodiments. For purposes of the specification, unless otherwise specified, the term “substantially” refers to the complete or nearly complete extent or degree of an action, characteristic, property, state, structure, item, or result. For example, in one embodiment, an object that is “substantially” located within a housing would mean that the object is either completely within a housing or nearly completely within a housing. The exact allowable degree of deviation from absolute completeness may in some cases depend on the specific context. However, generally speaking, the nearness of completion will be so as to have the same overall result as if absolute and total completion were obtained. The use of “substantially” is also equally applicable when used in a negative connotation to refer to the complete or near complete lack of an action, characteristic, property, state, structure, item, or result.

As used herein, the term “substantially,” in, for example, the context “substantially free” refers to a composition having less than about 10% by weight, e.g., less than about 5%, less than about 1%, less than about 0.5% by weight, less than about 0.1% by weight, less than about 0.05% by weight, or less than about 0.01% by weight of the stated material, based on the total weight of the composition.

It is further understood that the term “substantially,” when used in reference to a composition, refers to at least about 60% by weight, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% by weight, based on the total weight of the composition, of a specified feature, component, or a combination of the components. It is further understood that if the composition comprises more than one component, the two or more components may be present in any ratio predetermined by one of ordinary skill in the art. For example, and without limitation, the composition comprising substantially water and natural flavor, unless specifically recited, may comprise water and natural flavor in any ratio predetermined by one of ordinary skill in the art.

As used herein, the terms “approximately” and “about” generally refer to a deviance of within 5% of the indicated number or range of numbers. In one embodiment, the term “approximately” and “about”, may refer to a deviance of between 0.001-10% from the indicated number or range of numbers.

As used herein, the terms “electronic liquid,” “natural-based liquid composition,” or “e-liquid” may be used interchangeably and refer to a mixture used in a vapor product, such as an electronic vaporizing device. In some embodiments, an electronic vaporizing device may include without limitation electronic cigarettes, electronic pipes, electronic cigars, and the like.

As used herein, the term “emulsion” is directed to a fine dispersion of minute droplets of one liquid in another in which it is not soluble or miscible.

As used herein, the term “emulsifier” is directed to any substance capable of stabilizing an emulsion. It is understood that in some embodiments, the emulsifier prevents separation of the components present in the emulsion and results in a substantially homogeneous solution.

Disclosed are components that may be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all embodiments of this application including, but not limited to, steps in disclosed methods. Thus, if there are a variety of additional steps that may be performed it is understood that each of these additional steps may be performed with any specific embodiment or combination of embodiments of the disclosed methods.

The present methods and systems may be understood more readily by reference to the following detailed description of preferred embodiments and the examples included therein and to the Figures and their previous and following description.

As will be appreciated by one skilled in the art, the methods and systems may take the form of an entirely hardware embodiment, an entirely software embodiment, or an embodiment combining software and hardware embodiments. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, may be implemented by computer program instructions. These computer program instructions may be loaded onto a general-purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that may direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, may be implemented by special purpose hardware-based computer systems that perform the specified functions or steps, or combinations of special purpose hardware and computer instructions.

Various embodiments are now described with reference to the drawings. In the following description, for purposes of explanation, numerous specific details are set forth in order to provide a thorough understanding of one or more embodiments. It may be evident, however, that the various embodiments may be practiced without these specific details. In other instances, well-known structures and devices are shown in block diagram form to facilitate describing these embodiments.

In various implementations, there may be provided a liquid composition comprised of carrier and nicotine derived from at least one non-tobacco nicotine source and may be used in an electronic vaporizing device. In a preferred embodiment, the liquid composition may be used in an electronic vaporizing device employing a cold vapor misting distribution device, method, and/or system. In one embodiment, the liquid composition may be a natural-based liquid composition comprising substantially all-natural ingredients. In one embodiment, the liquid composition may be comprised of at least water and nicotine derived from at least one non-tobacco nicotine source.

In one embodiment, there may be provided a liquid composition for use in electronic vaporizing devices. In a preferred embodiment, the liquid composition may be used in electronic vaporizing devices employing a cold vapor misting distribution component or method. In one embodiment, the liquid composition may be comprised of about 70 weight percent to about 99 weight percent of water, about 0.01 weight percent to about 10 weight percent of nicotine derived from at least one non-tobacco nicotine source. In a preferred embodiment, the liquid composition may be a natural-based liquid composition and may be substantially free of at least one of propylene glycol, vegetable glycerin, and combinations thereof.

In one embodiment, the liquid composition may be comprised of a carrier, wherein the liquid composition may be comprised of about 70 weight percent to about 99 weight percent of carrier. In one embodiment, the carrier may comprise at least one of water, propylene glycol, vegetable glycerin, and combinations thereof.

In one embodiment, the liquid composition may be a natural-based liquid composition. In one embodiment, the natural-based liquid composition may be comprised primarily of water, wherein the natural-based liquid composition may be comprised of about 70 weight percent to about 99 weight percent of water. In one embodiment, water may be present in an amount of about 75 weight percent, about 80 weight percent, about 85 weight percent, about 90 weight percent, about 95 weight percent, about 96 weight percent, about 97 weight percent, and about 98 weight percent. In yet another embodiment, water may be present in amount from about 70 weight percent to about 97 weight percent, from about 70 weight percent to about 90 weight percent, or from about 80 weight percent to about 95 weight percent.

In one embodiment, the natural-based liquid composition may comprise nicotine derived from at least one non-tobacco nicotine source. Nicotine, or (S)-3-(1-Methyl-2-pyrrolidinyl)pyridine, is colorless alkaloid chemical found in the nightshade family of plants (Solanaceae). The majority of commercially used nicotine is obtained from tobacco plants. Unfortunately, however, the tobacco plant also contains many other components that are detrimental to humans, including, for example, known carcinogens and respiratory toxicants. The most harmful chemicals are tobacco-specific nitrosamines, which form during the growing curing, fermenting, and aging of tobacco. Therefore, the natural-based liquid composition may comprise nicotine derived from non-tobacco sources. In one embodiment, the at least one non-tobacco nicotine source may be selected from the Solanaceae plant family. In a preferred embodiment, the at least one non-tobacco nicotine source may be selected from the group of non-tobacco nicotine sources consisting of at least one of duboisia hopwoodii, eggplant, tomatoes, potatoes, green peppers, coca, and combinations thereof. In a more preferred embodiment, the at least one non-tobacco nicotine source may be duboisia hopwoodii.

In one embodiment, the at least one non-tobacco nicotine source may be present in an amount from about 0.01 weight percent to about 10 weight percent, including exemplary values of about 0.05 weight percent, about 0.1 weight percent, about 0.3 weight percent, about 0.5 weight percent, about 1 weight percent, about 2 weight percent, about 3 weight percent, about 4 weight percent, about 5 weight percent, about 6 weight percent, about 7 weight percent, about 8 weight percent, and about 9 weight percent. In still other embodiments, the at least one non-tobacco nicotine source may be present in any amount that falls between any foregoing values. In yet other embodiments, the at least one non-tobacco nicotine source may be present in an amount from about 0.01 weight percent to about 0.3 weight percent, from about 0.1 weight percent to about 5 weight percent, from about 1 weight percent to about 10 weight percent.

In one embodiment, the natural-based liquid composition may further comprise about 0.01 weight percent to about 10 weight percent of nornicotine derived from at least one non-tobacco nornicotine source. Nornicotine is an alkaloid that is chemically similar to nicotine, but does not contain a methyl group. The at least one non-tobacco nornicotine source may be from the same source as the at least one non-tobacco nicotine source or from a different source than the at least one non-tobacco nicotine source. In a preferred embodiment, the at least one non-tobacco nicotine source and the at least one non-tobacco nornicotine source may be the same source.

In one embodiment, the at least one non-tobacco nornicotine source may be present in an amount from about 0.01 weight percent to about 10 weight percent, including exemplary values of about 0.05 weight percent, about 0.1 weight percent, about 0.3 weight percent, about 0.5 weight percent, about 1 weight percent, about 2 weight percent, about 3 weight percent, about 4 weight percent, about 5 weight percent, about 6 weight percent, about 7 weight percent, about 8 weight percent, and about 9 weight percent. In still other embodiments, the at least one non-tobacco nornicotine source may be present in any amount that falls between any foregoing values. In yet other embodiments, the at least one non-tobacco nornicotine source may be present in an amount from about 0.01 weight percent to about 0.3 weight percent, from about 0.1 weight percent to about 5 weight percent, from about 1 weight percent to about 10 weight percent.

In one embodiment, the natural-based liquid composition may comprise at least one natural flavoring agent comprising a flavor of fruits, berries, spices, herbs, savory flavors, spicy flavors, sweet flavors, plant based flavors, and combinations thereof. In certain embodiments, the natural flavorings may comprise at least one of citrus flavorings, fruit flavorings, berry, spice flavorings, flower flavorings, herbaceous flavorings, vegetable flavorings, savory flavorings, sour flavorings, spicy flavorings, bitter flavorings, and combinations thereof. In yet other embodiments, the natural flavorings may comprise an essence of fruits, vegetables, flowers, spices, alcoholic beverages, or any combinations thereof. In still further embodiments, the natural-based liquid composition may comprise a manufacturing flavor derived from any natural ingredient that is known to have a pleasant flavor. It is understood that the list of flavorings is not limiting and any flavorings may comprise any component that is known to provide a pleasant taste to the user.

In one embodiment, the natural-based liquid composition may be comprised of about 1 weight percent to about 30 weight percent of at least one flavoring agent. In one embodiment, the at least one flavoring agent may be present in an amount of about 3 weight percent, about 5 weight percent, about 10 weight percent, about 15 weight percent, about 20 weight percent, and about 25 weight percent. In yet another embodiment, the at least one flavoring agent may be present in amount from about 3 weight percent to about 25 weight percent, from about 5 weight percent to about 25 weight percent, or from about 10 weight percent to about 30 weight percent.

In one embodiment, the natural-based liquid composition may further comprise at least one natural emulsifier. As one of ordinary skill in the art would readily appreciate, in some embodiments, an emulsifier may be needed to blend ingredients which would otherwise stay immiscible in a liquid due to their chemical composition. In one embodiment, the natural-based liquid composition is a substantially homogeneous composition. In one embodiment, the natural emulsifier disclosed herein may be any emulsifier capable of stabilizing an emulsion. In a preferred embodiment, the natural emulsifier may comprise a lecithin, a natural starch, a sunflower, or a combination thereof. It is understood that the lecithin may come from various sources. In certain embodiments, the lecithin may be derived from a plant, an animal, or combinations thereof. In one embodiment, the plant-based lecithin may comprise lecithin derived from sunflower, soybeans, rapeseeds, cottonseeds, or any combination thereof. In another embodiment, the animal-based lecithin may comprise lecithin derived from eggs, milk, marine animals, or any combination thereof.

In a preferred embodiment, the emulsifier may be derived from a sunflower plant. The direct portion of the sunflower plant, which contains properties of an emulsifier, is removed and added to the liquid composition. As such, the natural-based liquid composition may comprise an emulsifier which is all natural and in effect, not labeled as an emulsifier but merely labeled as a sunflower. This may be very significant because chemical emulsifiers, such as polysorbate-80 and carboxymethycellulose, commonly used in packaged foods, drinks, etc. have been found to cause obesity and gut inflammation.

Lecithin is a naturally occurring emulsifier made up of about five smaller molecules. It has a backbone of glycerol that bonds up to three other molecules. Two of the bonded molecules are fatty acids, which are hydrophobic. They give lecithin a structure that is similar to fats or lipids. The third substance attached to the glycerol is phosphoric acid that has an amino alcohol attached called choline. The phosphate/amino alcohol end of lecithin is hydrophilic. Emulsifiers are molecules that contain both a hydrophilic, water loving, and hydrophobic, water hating, portion. Lecithin has these same properties.

In yet other embodiments, the natural emulsifier may be present in an amount from about 0.01 weight percent to about 20 weight percent, including exemplary values of about 0.05 weight percent, about 0.1 weight percent, about 0.3 weight percent, about 0.5 weight percent, about 1 weight percent, about 2 weight percent, about 3 weight percent, about 4 weight percent, about 5 weight percent, about 6 weight percent, about 7 weight percent, about 8 weight percent, about 9 weight percent, about 10 weight percent, about 11 weight percent, about 12 weight percent, about 13 weight percent, about 14 weight percent, about 15 weight percent, about 16 weight percent, about 17 weight percent, about 18 weight percent, and about 19 weight percent. In still other embodiments, the natural emulsifier may be present in any amount that falls between any foregoing values. In yet other embodiments, the natural emulsifier may be present in an amount from about 0.01 weight percent to about 0.3 weight percent, from about 0.1 weight percent to about 15 weight percent, from about 1 weight percent to about 20 weight percent. The presence of the natural emulsifiers may result in the homogeneous distribution of all components present in the e-liquid solutions or compositions described herein.

In certain embodiments, the natural-based liquid composition may be substantially free of at least one of propylene glycol and vegetable glycerin. In yet other embodiments, the natural-based liquid composition may be substantially free of both propylene glycol and vegetable glycerin.

In yet other embodiments, the natural-based liquid composition may comprise less than about 95 weight percent of propylene glycol, less than about 90 weight percent of propylene glycol, less than about 85 weight percent of propylene glycol, less than about 80 weight percent of propylene glycol, less than about 75 weight percent of propylene glycol, less than about 70 weight percent of propylene glycol, less than about 65 weight percent of propylene glycol, less than about 60 weight percent of propylene glycol, less than about 55 weight percent of propylene glycol, less than about 50 weight percent of propylene glycol, less than about 45 weight percent of propylene glycol, less than about 40 weight percent of propylene glycol, less than about 35 weight percent of propylene glycol, less than about 30 weight percent of propylene glycol, less than about 25 weight percent of propylene glycol, less than about 20 weight percent of propylene glycol, less than about 15 weight percent of propylene glycol, less than about 10 weight percent of propylene glycol, and less than about 5 weight percent of propylene glycol.

In yet other embodiments, the natural-based liquid composition may comprise less than about 95 weight percent of vegetable glycerin, less than about 90 weight percent of vegetable glycerin, less than about 85 weight percent of vegetable glycerin, less than about 80 weight percent of vegetable glycerin, less than about 75 weight percent of vegetable glycerin, less than about 70 weight percent of vegetable glycerin, less than about 65 weight percent of vegetable glycerin, less than about 60 weight percent of vegetable glycerin, less than about 55 weight percent of vegetable glycerin, less than about 50 weight percent of vegetable glycerin, less than about 45 weight percent of vegetable glycerin, less than about 40 weight percent of vegetable glycerin, less than about 35 weight percent of vegetable glycerin, less than about 30 weight percent of vegetable glycerin, less than about 25 weight percent of vegetable glycerin, less than about 20 weight percent of vegetable glycerin, less than about 15 weight percent of vegetable glycerin, less than about 10 weight percent of vegetable glycerin, and less than about 5 weight percent of vegetable glycerin.

In other embodiments, the natural-based liquid composition may comprise less than about 95 weight percent of propylene glycol and vegetable glycerin, less than about 90 weight percent of propylene glycol and vegetable glycerin, less than about 85 weight percent of propylene glycol and vegetable glycerin, less than about 80 weight percent of propylene glycol and vegetable glycerin, less than about 75 weight percent of propylene glycol and vegetable glycerin, less than about 70 weight percent of propylene glycol and vegetable glycerin, less than about 65 weight percent of propylene glycol and vegetable glycerin, less than about 60 weight percent of propylene glycol and vegetable glycerin, less than about 55 weight percent of propylene glycol and vegetable glycerin, less than about 50 weight percent of propylene glycol and vegetable glycerin, less than about 45 weight percent of propylene glycol and vegetable glycerin, less than about 40 weight percent of propylene glycol and vegetable glycerin, less than about 35 weight percent of propylene glycol and vegetable glycerin, less than about 30 weight percent of propylene glycol and vegetable glycerin, less than about 25 weight percent of propylene glycol and vegetable glycerin, less than about 20 weight percent of propylene glycol and vegetable glycerin, less than about 15 weight percent of propylene glycol and vegetable glycerin, less than about 10 weight percent of propylene glycol and vegetable glycerin, and less than about 5 weight percent of propylene glycol and vegetable glycerin.

In certain embodiments, the natural-based liquid composition may comprise at least one supplementary component comprising a medicinal agent or element, a wellness agent or element, a recreational use agent or element, and any combinations of thereof.

In yet other embodiments, the wellness element may comprise chamomile, Echinacea, at least one homeopathic remedy, a vitamin supplement, or any combination thereof. In certain exemplary embodiments, the homeopathic remedy may comprise one or more of Abies Nigra, Carbo vegetablilis, Nux vomica, Robinia pseudoacacia, Arnica Montana, Bryonia, Dulcamara, Pulsatilla, Rhododendron chrysanthum, Rhus tox, Aesculus hippocastanum, Collinsonia Canadensis, Hamamelis virginiana, Phytolacca decandra, Rheum officinale, Dulcamara, Hydrastis Canadensis, Colocynthis, Allium cepa, Apis mellifica, Belladonna Eupatorium perfoliatum, Gelsemium sempervirens, Phytolacca decandra, Pulsatilla; Cimicifuga racemosa, Lycopodium clavatum, Nux moschata, Raphanus sativus, Calendula officinalis, Cineraria maritime, Euphrasia officinalis, Hyoscyamus niger, Nux moschata Passiflora incarnate, Stramonium, Anas barbariae, Arum triphyllum, Belladonna, Phytolacca decandra, Pulsatilla, Spongia tosta, Allium cepa, Ambrosia artemisiaefolia, Sabadilla, Solidago virgaurea, Aconitum napellus, Chelidonium majus, Jequirity, Viburnum opulus, Sanguinaria canadensis, Spigelia anthelmia, or any combination thereof.

In yet other embodiments, the wellness element may comprise any ancient and modern indigenous people health formulas. In some exemplary embodiments, the formulas may comprise tobacco, sweetgrass, sage, cedar, laurel, caraway, thyme, and the like. In yet other exemplary embodiments, the formulas may include various portions of a plant, for example and without limitation, the formulas may include seeds, berries, roots, leaves, fruits, bark, flowers, or the whole plant. It is understood that ancient and modern indigenous people health formulas include without limitation formulas utilized in the traditional Chinese, Korean, Japanese, Native American, Middle Eastern, European, Nepal, Aborigine, African, Western Pacific medicine and combinations thereof.

In one embodiment, the wellness element may be present in an amount from about 0.01 weight percent to about 10 weight percent, including exemplary values of about 0.05 weight percent, about 0.1 weight percent, about 0.3 weight percent, about 0.5 weight percent, about 1 weight percent, about 2 weight percent, about 3 weight percent, about 4 weight percent, about 5 weight percent, about 6 weight percent, about 7 weight percent, about 8 weight percent, and about 9 weight percent. In still other embodiments, the wellness element may be present in any amount that falls between any foregoing values. In yet other embodiments, the wellness element may be present in an amount from about 0.01 weight percent to about 0.3 weight percent, from about 0.1 weight percent to about 5 weight percent, from about 1 weight percent to about 10 weight percent.

In certain embodiments, the medicinal element may comprise diabetes medication, respiratory medication, sexual dysfunction remedy, cannabis based medication, or any combination thereof. It is further understood that if a combination of medicinal elements is used, the elements present in the natural-based liquid composition do not present harmful interactions with respect to the health of a user. It is further understood that any medications that may be configured for delivery through the mouth or lungs via the cold vaping system may be utilized.

In one embodiment, the medicinal element may be present in an amount from about 0.01 weight percent to about 10 weight percent, including exemplary values of about 0.05 weight percent, about 0.1 weight percent, about 0.3 weight percent, about 0.5 weight percent, about 1 weight percent, about 2 weight percent, about 3 weight percent, about 4 weight percent, about 5 weight percent, about 6 weight percent, about 7 weight percent, about 8 weight percent, and about 9 weight percent. In still other embodiments, the medicinal element may be present in any amount that falls between any foregoing values. In another embodiment, the medicinal element may be present in an amount from about 0.01 weight percent to about 0.3 weight percent, from about 0.1 weight percent to about 5 weight percent, from about 1 weight percent to about 10 weight percent.

In some embodiments, the recreational element may comprise caffeine, cannabis-based elements or compounds such as cannabinoids, taurine, salvia, kratum, kava, or any combination thereof. In another embodiment, the recreational element may be present in an amount from about 0.01 weight percent to about 10 weight percent, including exemplary values of about 0.05 weight percent, about 0.1 weight percent, about 0.3 weight percent, about 0.5 weight percent, about 1 weight percent, about 2 weight percent, about 3 weight percent, about 4 weight percent, about 5 weight percent, about 6 weight percent, about 7 weight percent, about 8 weight percent, and about 9 weight percent. In still other embodiments, the recreational element may be present in any amount that falls between any foregoing values. In yet other embodiments, the recreational element may be present in an amount from about 0.01 weight percent to about 0.3 weight percent, from about 0.1 weight percent to about 5 weight percent, from about 1 weight percent to about 10 weight percent.

In some embodiments, additional elements may be added to the natural-based liquid composition. In some embodiments, these elements comprise, for example, one or more additives and/or chemicals found in traditional cigarettes. For example, and without limitation, the elements may comprise Acetanisole, Acetic Acid, Acetoin, Acetophenone, 6-Acetoxydihydrotheaspirane, 2-Acetyl-3-Ethylpyrazine, 2-Acetyl-5-Methylfuran, Acetylpyrazine, 2-Acetylpyridine, 3-Acetylpyridine, 2-Acetylthiazole, Aconitic Acid, dl-Alanine, Alfalfa Extract, Allspice Extract, Oleoresin, and Oil, Allyl Hexanoate, Allyl Ionone, Almond Bitter Oil, Ambergris Tincture, Ammonia, Ammonium Bicarbonate, Ammonium Hydroxide, Ammonium Phosphate Dibasic, Ammonium Sulfide, Amyl Alcohol, Amyl Butyrate, Amyl Formate, Amyl Octanoate, alpha-Amylcinnamaldehyde, Amyris Oil, trans-Anethole, Angelica Root Extract, Oil and Seed Oil, Anise, Anise Star, Extract and Oils, Anisyl Acetate, Anisyl Alcohol, Anisyl Formate, Anisyl Phenylacetate, Apple Juice Concentrate, Extract, and Skins, Apricot Extract and Juice Concentrate, 1-Arginine, Asafetida Fluid Extract And Oil, Ascorbic Acid, 1-Asparagine Monohydrate, 1-Aspartic Acid, Balsam Peru and Oil, Basil Oil, Bay Leaf, Oil and Sweet Oil, Beeswax White, Beet Juice Concentrate, Benzaldehyde, Benzaldehyde Glyceryl Acetal, Benzoic Acid, Benzoin, Benzoin Resin, Benzophenone, Benzyl Alcohol, Benzyl Benzoate, Benzyl Butyrate, Benzyl Cinnamate, Benzyl Propionate, Benzyl Salicylate, Bergamot Oil, Bisabolene, Black Currant Buds Absolute, Borneo′, Bornyl Acetate, Buchu Leaf Oil, 1,3-Butanediol, 2,3-Butanedione, 1-Butanol, 2-Butanone, 4(2-Butenylidene)-3,5,5-Trimethyl-2-Cyclohexen-1-One, Butter, Butter Esters, and Butter Oil, Butyl Acetate, Butyl Butyrate, Butyl Butyryl Lactate, Butyl Isovalerate, Butyl Phenylacetate, Butyl Undecylenate, 3-Butylidenephthalide, Butyric Acid, Cadinene, Caffeine, Calcium Carbonate, Camphene, Cananga Oil, Capsicum Oleoresin, Caramel Color, Caraway Oil, Carbon Dioxide, Cardamom Oleoresin, Extract, Seed Oil, and Powder, Carob Bean and Extract, beta-Carotene, Carrot Oil, Carvacrol, 4-Carvomenthenol, 1-Carvone, beta-Caryophyllene, beta-Caryophyllene Oxide, Cascarilla Oil and Bark Extract, Cassia Bark Oil, Cassie Absolute and Oil, Castoreum Extract, Tincture and Absolute, Cedar Leaf Oil, Cedarwood Oil Terpenes and Virginiana, Cedrol, Celery Seed Extract, Solid, Oil, And Oleoresin, Cellulose Fiber, Chamomile Flower Oil And Extract, Chicory Extract, Chocolate, Cinnamaldehyde, Cinnamic Acid, Cinnamon leaf Oil, Bark Oil, and Extract, Cinnamyl Acetate, Cinnamyl Alcohol, Cinnamyl Cinnamate, Cinnamyl Isovalerate, Cinnamyl Propionate, Citral, Citric Acid, Citronella Oil, dl-Citronellol, Citronellyl Butyrate, Citronellyl Isobutyrate, Civet Absolute, Clary Oil, Clover Tops, Red Solid Extract, Cocoa, Cocoa Shells, Extract, Distillate And Powder, Coconut Oil, Coffee, Cognac White and Green Oil, Copaiba Oil, Coriander Extract and Oil, Corn Oil, Corn Silk, Costus Root Oil, Cubeb Oil, Cuminaldehyde, para-Cymene, 1-Cysteine, Dandelion Root Solid Extract, Davana Oil, 2-trans, 4-trans-Decadienal, delta-Decalactone, gamma-Decalactone, Decanal, Decanoic Acid, 1-Decanol, 2-Decenal, Dehydromenthofurolactone, Diethyl Malonate, Diethyl Sebacate, 2,3-Diethylpyrazine, Dihydro Anethole, 5,7-Dihydro-2-Methylthieno(3,4-D) Pyrimidine, Dill Seed Oil and Extract, meta-Dimethoxybenzene, para-Dimethoxybenzene, 2,6-Dimethoxyphenol, Dimethyl Succinate, 3,4-Dimethyl-1,2 Cyclopentanedione, 3,5-Dimethyl-1,2-Cyclopentanedione, 3,7-Dimethyl-1,3,6-Octatriene, 4,5-Dimethyl-3-Hydroxy-2,5-Dihydrofuran-2-One, 6,10-Dimethyl-5,9-Undecadien-2-One, 3,7-Dimethyl-6-Octenoic Acid, 2,4 Dimethylacetophenone, alpha,para-Dimethylbenzyl Alcohol, alpha,alpha-Dimethylphenethyl Acetate, alpha,alpha Dimethylphenethyl Butyrate, 2,3-Dimethylpyrazine, 2,5-Dimethylpyrazine, 2,6 DimethylpyrazinDimethyltetrahydrobenzofuranone, delta-Dodecalactone, gamma-Dodecalactone, Para-Ethoxybenzaldehyde, Ethyl 10-Undecenoate, Ethyl 2-Methylbutyrate, Ethyl Acetate, Ethyl Acetoacetate, Ethyl Alcohol, Ethyl Benzoate, Ethyl Butyrate, Ethyl Cinnamate, Ethyl Decanoate Fenchol, Ethyl Furoate, Ethyl Heptanoate, Ethyl Hexanoate, Ethyl Isovalerate, Ethyl Lactate, Ethyl Laurate, Ethyl Levulinate, Ethyl Maltol, Ethyl Methyl Phenylglycidate, Ethyl Myristate, Ethyl Nonanoate, Ethyl Octadecanoate, Ethyl Octanoate, Ethyl Oleate, Ethyl Palmitate, Ethyl Phenylacetate, Ethyl Propionate, Ethyl Salicylate, Ethyl trans-2-Butenoate, Ethyl Valerate, Ethyl Vanillin, 2-Ethyl (or Methyl)-(3,5 and 6)-Methoxypyrazine, 2-Ethyl-1-Hexanol, 3-Ethyl-2-Hydroxy-2-Cyclopenten-1-One, 2-Ethyl-3, (5 or 6)-Dimethylpyrazine, 5-Ethyl-3-Hydroxy-4-Methyl-2(5H)-Furanone, 2-Ethyl-3-Methylpyrazine, 4-Ethylbenzaldehyde, 4-Ethylguaiacol, para-Ethylphenol, 3-Ethylpyridine, Eucalyptol, Farnesol, D-Fenchone, Fennel Sweet Oil, Fenugreek, Extract, Resin, and Absolute, Fig Juice Concentrate, Food Starch Modified, Furfuryl Mercaptan, 4-(2-Furyl)-3-Buten-2-One, Galbanum Oil, Genet Absolute, Gentian Root Extract, Geraniol, Geranium Rose Oil, Geranyl Acetate, Geranyl Butyrate, Geranyl Formate, Geranyl Isovalerate, Geranyl Phenylacetate, Ginger Oil and Oleoresin, 1-Glutamic Acid, 1-Glutamine, Glycerol, Glycyrrhizin Ammoniated, Grape Juice Concentrate, Guaiac Wood Oil, Guaiacol, Guar Gum, 2,4-Heptadienal, gamma-Heptalactone, Heptanoic Acid, 2-Heptanone, 3-Hepten-2-One, 2-Hepten-4-One, 4-Heptenal, trans-2-Heptenal, Heptyl Acetate, omega-6-Hexadecenlactone, gamma-Hexalactone, Hexanal, Hexanoic Acid, 2-Hexen-1-Ol, 3-Hexen-1-01, cis-3-Hexen-1-Yl Acetate, 2-Hexenal, 3-Hexenoic Acid, trans-2-Hexenoic Acid, cis-3-Hexenyl Formate, Hexyl 2-Methylbutyrate, Hexyl Acetate, Hexyl Alcohol, Hexyl Phenylacetate, 1-Histidine, Honey, Hops Oil, Hydrolyzed Milk Solids, Hydrolyzed Plant Proteins, 5-Hydroxy-2,4-Decadienoic Acid delta-Lactone, 4-Hydroxy-2,5-Dimethyl-3 (2H)-Furanone, 2-Hydroxy-3,5,5-Trimethyl-2-Cyclohexen-1-One, 4-Hydroxy-3-Pentenoic Acid Lactone, 2-Hydroxy-4-Methylbenzaldehyde, 4-Hydroxybutanoic Acid Lactone, Hydroxycitronellal, 6-Hydroxydihydrotheaspirane, 4-(para-Hydroxyphenyl)-2-Butanone, Hyssop Oil, Immortelle Absolute and Extract, alpha-Ionone, beta-Ionone, alpha-Irone, Isoamyl Acetate, Isoamyl Benzoate, Isoamyl Butyrate, Isoamyl Cinnamate, Isoamyl Formate, Isoamyl Hexanoate, Isoamyl Isovalerate, Isoamyl Octanoate, Isoamyl Phenylacetate, Isobornyl Acetate, Isobutyl Acetate, Isobutyl Alcohol, Isobutyl Cinnamate, Isobutyl Phenylacetate, Isobutyl Salicylate, 2-Isobutyl-3-Methoxypyrazine, alpha-Isobutylphenethyl Alcohol, Isobutyraldehyde, Isobutyric Acid, d,l-Isoleucine, alpha-Isomethylionone, 2-Isopropylphenol, Isovaleric Acid, Jasmine Absolute, Concrete and Oil, Kola Nut Extract, Labdanum Absolute and Oleoresin, Lactic Acid, Lauric Acid, Lauric Aldehyde, Lavandin Oil, Lavender Oil, Lemon Oil and Extract, Lemongrass Oil, 1-Leucine, Levulinic Acid, Licorice Root, Fluid, Extract and Powder, Lime Oil, Linalool, Linalool Oxide, Linalyl Acetate, Linden Flowers, Lovage Oil And Extract, 1-Lysine, Mace Powder, Extract and Oil, Magnesium Carbonate, Malic Acid, Malt and Malt Extract, Maltodextrin, Maltol, Maltyl Isobutyrate, Mandarin Oil, Maple Syrup and Concentrate, Mate Leaf, Absolute and Oil, para-Mentha-8-Thiol-3-One, Menthol, Menthone, Menthyl Acetate, dl-Methionine, Methoprene, 2-Methoxy-4-Methylphenol, 2-Methoxy-4-Vinylphenol, para-Methoxybenzaldehyde, 1-(para-Methoxyphenyl)-1-Penten-3-One, 4-(para-Methoxyphenyl)-2-Butanone, 1-(para-Methoxyphenyl)-2-Propanone, Methoxypyrazine, Methyl 2-Furoate, Methyl 2-Octynoate, Methyl 2-Pyrrolyl Ketone, Methyl Anisate, Methyl Anthranilate, Methyl Benzoate, Methyl Cinnamate, Methyl Dihydrojasmonate, Methyl Ester of Rosin, Partially Hydrogenated, Methyl Isovalerate, Methyl Linoleate (48%), Methyl Linolenate (52%) Mixture, Methyl Naphthyl Ketone, Methyl Nicotinate, Methyl Phenylacetate, Methyl Salicylate, Methyl Sulfide, 3-Methyl-1-Cyclopentadecanone, 4-Methyl-1-Phenyl-2-Pentanone, 5-Methyl-2-Phenyl-2-Hexenal, 5-Methyl-2-Thiophenecarboxaldehyde, 6-Methyl-3,-5-Heptadien-2-One, 2-Methyl-3-(para-Isopropylphenyl) Propionaldehyde, 5-Methyl-3-Hexen-2-One, 1-Methyl-3Methoxy-4-Isopropylbenzene, 4-Methyl-3-Pentene-2-One, 2-Methyl-4-Phenylbutyraldehyde, 6-Methyl-5-Hepten-2-One, 4-Methyl-5-Thiazoleethanol, 4-Methyl-5-Vinylthiazole, Methyl-trans-2-Butenoic Acid, 4-Methylacetophenone, para-Methylanisole, alpha-Methylbenzyl Acetate, alpha-Methylbenzyl Alcohol, 2-Methylbutyraldehyde, 3-Methylbutyraldehyde, 2-Methylbutyric Acid, alpha-Methylcinnamaldehyde, Methylcyclopentenolone, 2-Methylheptanoic Acid, 2-Methylhexanoic Acid, 3-Methylpentanoic Acid, 4-Methylpentanoic Acid, 2-Methylpyrazine, 5-Methylquinoxaline, 2-Methyltetrahydrofuran-3-One, (Methylthio)Methylpyrazine (Mixture Of Isomers), 3-Methylthiopropionaldehyde, Methyl 3-Methylthiopropionate, 2-Methylvaleric Acid, Mimosa Absolute and Extract, Molasses Extract and Tincture, Mountain Maple Solid Extract, Mullein Flowers, Myristaldehyde, Myristic Acid, Myrrh Oil, Beta-Napthyl Ethyl Ether, Nerol, Neroli Bigarde Oil, Nerolidol, Nona-2-trans, 6-cis-Dienal, 2,6-Nonadien-1-01, gamma-Nonalactone, Nonanal, Nonanoic Acid, Nonanone, trans-2-Nonen-1-Ol, 2-Nonenal, Nonyl Acetate, Nutmeg Powder and Oil, Oak Chips Extract and Oil, Oak Moss Absolute, 9,12-Octadecadienoic Acid (48%) And 9,12,15-Octadecatrienoic Acid (52%), delta-Octalactone, gamma-Octalactone, Octanal, Octanoic Acid, 1-Octanol, 2-Octanone, 3-Octen-2-One, 1-Octen-3-01, 1-Octen-3-Yl Acetate, 2-Octenal, Octyl Isobutyrate, Oleic Acid, Olibanum Oil, Opoponax Oil And Gum, Orange Blossoms Water, Absolute, and Leaf Absolute, Orange Oil and Extract, Origanum Oil, Orris Concrete Oil and Root Extract, Palmarosa Oil, Palmitic Acid, Parsley Seed Oil, Patchouli Oil, omega-Pentadecalactone, 2,3-Pentanedione, 2-Pentanone, 4-Pentenoic Acid, 2-Pentylpyridine, Pepper Oil, Black And White, Peppermint Oil, Peruvian (Bois De Rose) Oil, Petitgrain Absolute, Mandarin Oil and Terpeneless Oil, alpha-Phellandrene, 2-Phenenthyl Acetate, Phenenthyl Alcohol, Phenethyl Butyrate, Phenethyl Cinnamate, Phenethyl Isobutyrate, Phenethyl Isovalerate, Phenethyl Phenylacetate, Phenethyl Salicylate, 1-Phenyl-1-Propanol, 3-Phenyl-1-Propanol, 2-Phenyl-2-Butenal, 4-Phenyl-3-Buten-2-01, 4-Phenyl-3-Buten-2-One, Phenylacetaldehyde, Phenylacetic Acid, 1-Phenylalanine, 3-Phenylpropionaldehyde, 3-Phenylpropionic Acid, 3-Phenylpropyl Acetate, 3-Phenylpropyl Cinnamate, 2-(3-Phenylpropyl)Tetrahydrofuran, Phosphoric Acid, Pimenta Leaf Oil, Pine Needle Oil, Pine Oil, Scotch, Pineapple Juice Concentrate, alpha-Pinene, beta-Pinene, D-Piperitone, Piperonal, Pipsissewa Leaf Extract, Plum Juice, Potassium Sorbate, 1-Proline, Propenylguaethol, Propionic Acid, Propyl Acetate, Propyl para-Hydroxybenzoate, Propylene Glycol, 3-Propylidenephthalide, Prune Juice and Concentrate, Pyridine, Pyroligneous Acid And Extract, Pyrrole, Pyruvic Acid, Raisin Juice Concentrate, Rhodinol, Rose Absolute and Oil, Rosemary Oil, Rum, Rum Ether, Rye Extract, Sage, Sage Oil, and Sage Oleoresin, Salicylaldehyde, Sandalwood Oil, Yellow, Sclareolide, Skatole, Smoke Flavor, Snakeroot Oil, Sodium Acetate, Sodium Benzoate, Sodium Bicarbonate, Sodium Carbonate, Sodium Chloride, Sodium Citrate, Sodium Hydroxide, Solanone, Spearmint Oil, Styrax Extract, Gum and Oil, Sucrose Octaacetate, Sugar Alcohols, Sugars, Tagetes Oil, Tannic Acid, Tartaric Acid, Tea Leaf and Absolute, alpha-Terpineol, Terpinolene, Terpinyl Acetate, 5,6,7,8-Tetrahydroquinoxaline, 1,5,5,9-Tetramethyl-13-Oxatricyclo(8.3.0.0(4,9))Tridecane, 2,3,4,5, and 3,4,5,6-Tetramethylethyl-Cyclohexanone, 2,3,5,6-Tetramethylpyrazine, Thiamine Hydrochloride, Thiazole, 1-Threonine, Thyme Oil, White and Red, Thymol, Tobacco Extracts, Tochopherols (mixed), Tolu Balsam Gum and Extract, Tolualdehydes, para-Tolyl 3-Methylbutyrate, para-Tolyl Acetaldehyde, para-Tolyl Acetate, para-Tolyl Isobutyrate, para-Tolyl Phenylacetate, Triacetin, 2-Tridecanone, 2-Tridecenal, Triethyl Citrate, 3,5,5-Trimethyl-1-Hexanol, para,alpha,alpha-Trimethylbenzyl Alcohol, 4-(2,6,6-Trimethylcyclohex-1-Enyl)But-2-En-4-One, 2,6,6-Trimethylcyclohex-2-Ene-1,4-Dione, 2,6,6-Trimethylcyclohexa-1,3-Dienyl Methane, 4-(2,6,6-Trimethylcyclohexa-1,3-Dienyl)But-2-En-4-One, 2,2,6-Trimethylcyclohexanone, 2,3,5-Trimethylpyrazine, 1-Tyrosine, Delta-Undercalactone, Gamma-Undecalactone, Undecanal, 2-Undecanone, 1, 0-Undecenal, Urea, Valencene, Valeraldehyde, Valerian Root Extract, Oil and Powder, Valeric Acid, gamma-Valerolactone, Valine, Vanilla Extract And Oleoresin, Vanillin, Veratraldehyde, Vetiver Oil, Vinegar, Violet Leaf Absolute, Walnut Hull Extract, Water, Wheat Extract And Flour, Wild Cherry Bark Extract, Wine and Wine Sherry, Xanthan Gum, 3,4-Xylenol, and Yeast. It is further understood that these elements may mimic the feel of real cigarettes to aid in enabling people to stop smoking.

In some embodiments, if a decreased amount of recreational elements, or elements mimicking the feel of real cigarettes are required, natural compounds capable of mimicking the sensory experience (e.g. taste, smell, etc.) associated with consumption (e.g. inhalation, absorption) of the element may be added. For example, if there is a desire to reduce the amount of recreational elements, the natural-based liquid composition may comprise an increased concentration of mint, mimicking the sensation of the menthol. In some embodiments, a decrease in the concentration of the specific element may require an increase in more than one natural component.

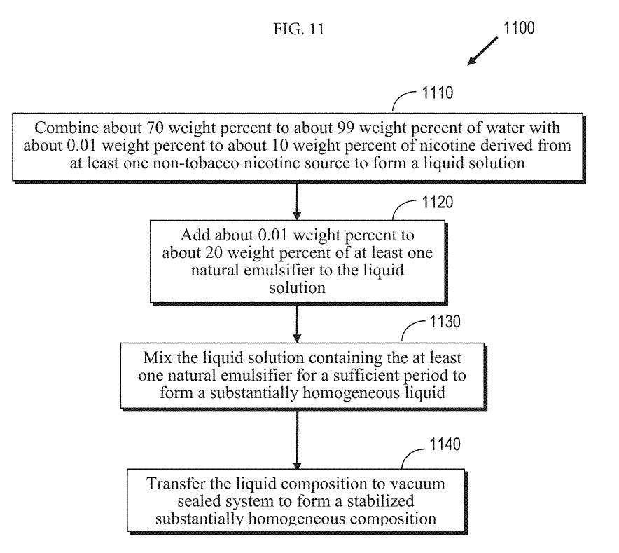

In an embodiment, as illustrated in FIG. 11, a method 1100 may be provided for producing a natural-based liquid composition configured for use in an electronic vaporizing device. The method may comprise the step 1110 of combining about 70 weight percent to about 99 weight percent of water with about 0.01 weight percent to about 10 weight percent of nicotine derived from at least one non-tobacco nicotine source to form a liquid solution.

In one embodiment, the at least one non-tobacco nicotine source may be selected from the Solanaceae plant family. In a preferred embodiment, the at least one non-tobacco nicotine source may be selected from the group of non-tobacco nicotine sources consisting of at least one of duboisia hopwoodii, eggplant, tomatoes, potatoes, green peppers, coca, and combinations thereof. In a more preferred embodiment, the at least one non-tobacco nicotine source may be duboisia hopwoodii.

The at least one non-tobacco nicotine source may be subjected to any suitable process to derive, extract, separate, or isolate nicotine therefrom for use in the natural-based liquid composition. In one embodiment, the at least one non-tobacco nicotine source material may be brought into contact with an extraction solvent under conditions to extract at least a portion of the nicotine therefrom. The extract may then be filtered to remove the solid or larger particles of the non-tobacco nicotine source material from the liquid extract. The liquid extract may then be further processed as required for addition to the natural-based liquid composition. In one embodiment, the extraction solvent may be selected from at least one of carbon dioxide, isopropyl alcohol, propane, butane, hexane, and combinations thereof.

The method may further comprise the step 1120 of adding about 0.01 weight percent to about 20 weight percent of at least one natural emulsifier to the liquid solution. In one embodiment, the at least one natural emulsifier is selected from the group comprising at least one of a sunflower, a lecithin, a natural starch, and combinations thereof.

The method may comprise the step 1130 of mixing the liquid solution containing the at least one natural emulsifier for a sufficient period to form a substantially homogeneous liquid composition. The natural-based liquid composition so produced may be substantially free of propylene glycol, vegetable glycerin, or both.

The method may further comprise the step 1140 of transferring the substantially homogeneous liquid composition to vacuum sealed system to form a stabilized substantially homogeneous composition.

The method may also comprise adding about 0.01 weight percent to about 10 weight percent of at least one supplementary component to the liquid solution, wherein the at least one supplementary component is selected from the group comprising at least one of a flavoring agent, a medicinal agent, a wellness agent, a recreational use agent, nornicotine and combinations thereof.

In another embodiment, the electronic vaporizing device may be a hybrid component of a mobile electronic communication device, such as a cellular telephone, electronic tablet device, or personal computing device. The electronic vaporizing device may be a single rechargeable component, continuous with the electronic communication device, or may be portable, disposable or recyclable, removable, and combinations thereof. The devices may be either hard wired together via an electronic connection at the edges of the devices, and in some instances, with some designed overlap, depending on how the devices will optimize continuity and function. The metallic electrical connections between devices may be flush, or at least one of the devices may deploy internal connections into the other device as are known in the art. In one embodiment, the devices are connected via a protruding port penetrating precise fit positioning of the electrical connection leading from one device inside the other device to engage the electrical connection. A locking system may keep the two parts firmly in place, as though the two devices were one. In some embodiments, the device described herein may be configured be delivered in a companion cartridge. In yet other embodiments, the companion cartridge may comprise a refillable cartridge, a disposable cartridge, a cartridge configured for independent use, a companion device, or a rechargeable cartridge.

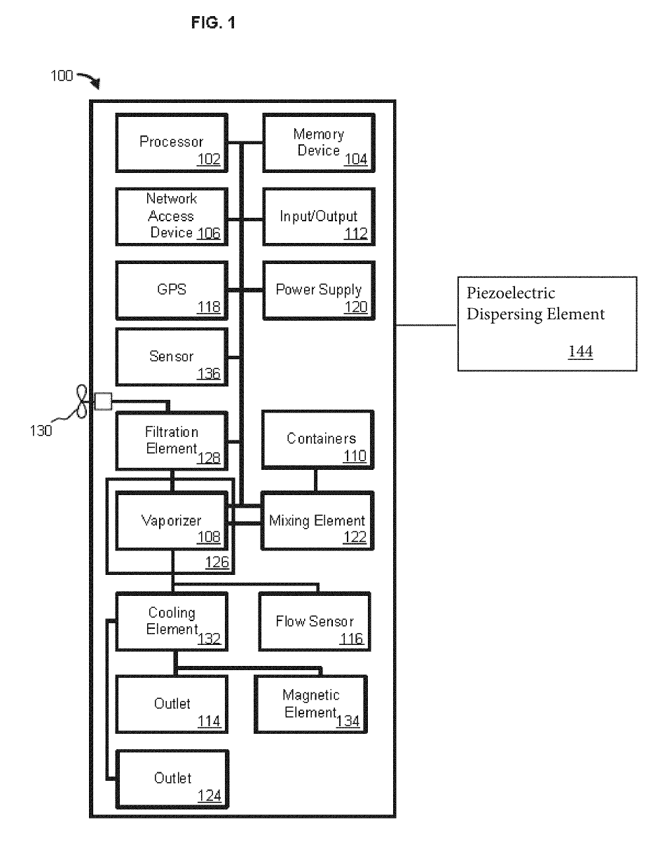

FIG. 1 is a block diagram of one embodiment of an electronic vaporizing device 100 as described herein. The electronic vaporizing device 100 may be, for example, an electronic cigarette, an electronic cigar, an electronic vapor device, a hybrid electronic communication device coupled/integrated vapor device, a robotic vapor device, a modified vapor device (also known as a mod), a micro-sized electronic vapor device, and the like. The electronic vaporizing device 100 may comprise any suitable housing for enclosing and protecting the various components disclosed herein. The electronic vaporizing device 100 may comprise a processor 102 operable to control the operation of the electronic vaporizing device 100. The processor 102 may be, or may comprise, any suitable microprocessor or microcontroller, for example, a low-power application-specific controller (ASIC) and/or a field programmable gate array (FPGA) designed or programmed specifically for the task of controlling a device as described herein, or a general purpose central processing unit (CPU), for example, one based on 80×86 architecture as designed by Intel™ or AMD™, or a system-on-a-chip as designed by ARM™. The processor 102 may be coupled (e.g., communicatively, operatively, etc.) to auxiliary devices or modules of the electronic vaporizing device 100 using a bus or other coupling. The electronic vaporizing device 100 may comprise power supply 120. The power supply 120 may comprise one or more batteries and/or other power storage device (e.g., capacitor) and/or a port for connecting to an external power supply. The one or more batteries may be rechargeable. The one or more batteries may comprise a lithium-ion battery (including thin film lithium ion batteries), a lithium-ion polymer battery, a nickel-cadmium battery, a nickel metal hydride battery, a lead-acid battery, combinations thereof, and the like. For example, an external power supply may supply power to the electronic vaporizing device 100 and a battery may store at least a portion of the supplied power.

The electronic vaporizing device 100 may comprise a memory device 104 coupled to the processor 102. The memory device 104 may comprise a random access memory (RAM) configured for storing program instructions and data for execution or processing by the processor 102 during control of the electronic vaporizing device 100. When the electronic vaporizing device 100 is powered off or in an inactive state, program instructions and data may be stored in a long-term memory, for example, a non-volatile magnetic optical, or electronic memory storage device (not shown). At least one of the RAM or the long-term memory may comprise a non-transitory computer-readable medium storing program instructions that, when executed by the processor 102, cause the electronic vaporizing device 100 to perform all or part of one or more methods and/or operations described herein. Program instructions may be written in any suitable high-level language, for example, C, C++, C# or the Java™, and compiled to produce machine-language code for execution by the processor 102.

In one embodiment, the electronic vaporizing device 100 may comprise a network access device 106 allowing the electronic vaporizing device 100 to be coupled to one or more ancillary devices (not shown) such as via an access point (not shown) of a wireless telephone network, local area network, or other coupling to a wide area network, for example, the Internet. In that regard, the processor 102 may be configured to share data with the one or more ancillary devices via the network access device 106. The shared data may comprise, for example, usage data and/or operational data of the electronic vaporizing device 100, a status of the electronic vaporizing device 100, a status and/or operating condition of one or more the components of the electronic vaporizing device 100, text to be used in a message, a product order, payment information, and/or any other data. Similarly, the processor 102 may be configured to receive control instructions from the one or more ancillary devices via the network access device 106. For example, a configuration of the electronic vaporizing device 100, an operation of the electronic vaporizing device 100, and/or other settings of the electronic vaporizing device 100, may be controlled by the one or more ancillary devices via the network access device 106. For example, an ancillary device may comprise a server that may provide various services and another ancillary device may comprise a smartphone for controlling operation of the electronic vaporizing device 100. In some embodiments, the smartphone or another ancillary device may be used as a primary input/output of the electronic vaporizing device 100 such that data may be received by the electronic vaporizing device 100 from the server, transmitted to the smartphone, and output on a display of the smartphone. In an embodiment, data transmitted to the ancillary device may comprise a mixture of vaporizable material and/or instructions to release vapor. For example, the electronic vaporizing device 100 may be configured to determine a need for the release of vapor into the atmosphere. The electronic vaporizing device 100 may provide instructions via the network access device 106 to an ancillary device (e.g., another vapor device) to release vapor into the atmosphere.

In an embodiment, the electronic vaporizing device 100 may also comprise an input/output device 112 coupled to one or more of the processor 102, the vaporizer 108, the network access device 106, and/or any other electronic component of the electronic vaporizing device 100. Input may be received from a user or another device and/or output may be provided to a user or another device via the input/output device 112. The input/output device 112 may comprise any combinations of input and/or output devices such as buttons, knobs, keyboards, touchscreens, displays, light-emitting elements, a speaker, and/or the like. In an embodiment, the input/output device 112 may comprise an interface port (not shown) such as a wired interface, for example a serial port, a Universal Serial Bus (USB) port, an Ethernet port, or other suitable wired connection. The input/output device 112 may comprise a wireless interface (not shown), for example a transceiver using any suitable wireless protocol, for example Wi-Fi (IEEE 802.11), Bluetooth®, infrared, or other wireless standard. For example, the input/output device 112 may communicate with a smartphone via Bluetooth® such that the inputs and outputs of the smartphone may be used by the user to interface with the electronic vaporizing device 100. In an embodiment, the input/output device 112 may comprise a user interface. The user interface may comprise at least one of lighted signal lights, gauges, boxes, forms, check marks, avatars, visual images, graphic designs, lists, active calibrations or calculations, 2D interactive fractal designs, 3D fractal designs, 2D and/or 3D representations of vapor devices and other interface system functions.

In an embodiment, the input/output device 112 may comprise a touchscreen interface and/or a biometric interface. For example, the input/output device 112 may include controls that allow the user to interact with and input information and commands to the electronic vaporizing device 100. For example, with respect to the embodiments described herein, the input/output device 112 may comprise a touch screen display. The input/output device 112 may be configured to provide the content of the exemplary screen shots shown herein, which are presented to the user via the functionality of a display. User inputs to the touch screen display are processed by, for example, the input/output device 112 and/or the processor 102. The input/output device 112 may also be configured to process new content and communications to the electronic vaporizing device 100. The touch screen display may provide controls and menu selections, and process commands and requests. Application and content objects may be provided by the touch screen display. The input/output device 112 and/or the processor 102 may receive and interpret commands and other inputs, interface with the other components of the electronic vaporizing device 100 as required. In an embodiment, the touch screen display may enable a user to lock, unlock, or partially unlock or lock, the electronic vaporizing device 100. The electronic vaporizing device 100 may be transitioned from an idle and locked state into an open state by, for example, moving or dragging an icon on the screen of the electronic vaporizing device 100, entering in a password/passcode, and the like. The input/output device 112 may thus display information to a user such as a puff count, an amount of vaporizable material remaining in the containers 110, battery remaining, signal strength, combinations thereof, and the like.

In an embodiment, the input/output device 112 may comprise an audio user interface. A microphone may be configured to receive audio signals and relay the audio signals to the input/output device 112. The audio user interface may be any interface that is responsive to voice or other audio commands. The audio user interface may be configured to cause an action, activate a function, etc., by the electronic vaporizing device 100 (or another device) based on a received voice (or other audio) command. The audio user interface may be deployed directly on the electronic vaporizing device 100 and/or via other electronic devices (e.g., electronic communication devices, such as a smartphone, a smart watch, a tablet, a laptop, a dedicated audio user interface device, other personal computing devices, and the like). The audio user interface may be used to control the functionality of the electronic vaporizing device 100. Such functionality may comprise, but is not limited to, custom mixing of vaporizable material (e.g., liquid compositions) and/or ordering custom made liquid composition combinations via an eCommerce service (e.g., specifications of a user's custom flavor mix may be transmitted to an eCommerce service, so that a liquid composition provider may mix a custom liquid composition cartridge for the user). The user may then reorder the custom flavor mix anytime or even send it to friends as a present, all via the audio user interface. The user may also send via voice command a mixing recipe to other users. The other users may utilize the mixing recipe (e.g., via an electronic vapor device having multiple chambers for natural-based liquid composition) to sample the same mix via an auto-order to the other users' devices to create the received mixing recipe. A custom mix may be given a title by a user and/or may be defined by parts (e.g., one part liquid A and two parts liquid B). The audio user interface may also be utilized to create and send a custom message to other users, to join electronic vaporizing clubs, to receive electronic vaporizing chart information, and to conduct a wide range of social networking, location services and eCommerce activities. The audio user interface may be secured via a password (e.g., audio password) which features at least one of tone recognition, other voice quality recognition and, in one embodiment, may utilize at least one special cadence as part of the audio password.

The input/output device 112 may be configured to interface with other devices, for example, exercise equipment, computing equipment, communications devices and/or other vapor devices, for example, via a physical or wireless connection. The input/output device 112 may thus exchange data with the other equipment. A user may sync their electronic vaporizing device 100 to other devices, via programming attributes such as mutual dynamic link library (DLL) ‘hooks’. This enables a smooth exchange of data between devices, as may a web interface between devices. The input/output device 112 may be used to upload one or more profiles to the other devices. Using exercise equipment as an example, the one or more profiles may comprise data such as workout routine data (e.g., timing, distance, settings, heart rate, etc.) and vaping data (e.g., natural-based liquid composition mixture recipes, supplements, vaping timing, etc.). Data from usage of previous exercise sessions may be archived and shared with new electronic vapor devices and/or new exercise equipment so that history and preferences may remain continuous and provide for simplified device settings, default settings, and recommended settings based upon the synthesis of current and archival data.

As shown in FIG. 1, in an embodiment, the electronic vaporizing device 100 may comprise a vaporizer 108. The vaporizer 108 may be coupled to one or more containers 110. Each of the one or more containers 110 may be configured to hold one or more vaporizable or non-vaporizable materials. The vaporizer 108 may receive the one or more vaporizable or non-vaporizable materials from the one or more containers 110 and heat the one or more vaporizable or non-vaporizable materials until the one or more vaporizable or non-vaporizable materials achieve a vapor state. In various embodiments, instead of heating the one or more vaporizable or non-vaporizable materials, the vaporizer 108 may nebulize or otherwise cause the one or more vaporizable or non-vaporizable materials in the one or more containers 110 to reduce in size into particulates. In various embodiments, the one or more containers 110 may comprise a compressed liquid that may be released to the vaporizer 108 via a valve or another mechanism. In various embodiments, the one or more containers 110 may comprise a wick (not shown) through which the one or more vaporizable or non-vaporizable materials is drawn to the vaporizer 108. The one or more containers 110 may be made of any suitable structural material, such as, an organic polymer, metal, ceramic, composite, or glass material.

In an embodiment, the electronic vaporizing device 100 may comprise a mixing element or component 122. The mixing component 122 may be coupled to the processor 102 to receive one or more mixing control signals. The one or more mixing control signals may instruct the mixing component 122 to withdraw specific amounts of material from the one or more containers 110. The mixing component 122 may, in response to a mixing control signal from the processor 102, withdraw select quantities of vaporizable material to create a customized mixture of different types of vaporizable material. The liquid withdrawn by the mixing component 122 may be provided to the vaporizer 108.

In an embodiment, input from the input/output device 112 may be used by the processor 102 to cause the vaporizer 108 to vaporize the one or more vaporizable or non-vaporizable materials. For example, a user may depress a button, causing the vaporizer 108 to start vaporizing or heating the one or more vaporizable or non-vaporizable materials. A user may then draw on an outlet 114 to inhale the vapor. In various embodiments, the processor 102 may control vapor production and flow to the outlet 114 based on data detected by a flow sensor 116. For example, as a user draws on the outlet 114, the flow sensor 116 may detect the resultant pressure and provide a signal to the processor 102. In response, the processor 102 may cause the vaporizer 108 to begin vaporizing the one or more vaporizable or non-vaporizable materials, terminate vaporizing the one or more vaporizable or non-vaporizable materials, and/or otherwise adjust a rate of vaporization of the one or more vaporizable or non-vaporizable materials. In another embodiment, the vapor may exit the electronic vaporizing device 100 through an outlet 124. The outlet 124 differs from the outlet 114 in that the outlet 124 may be configured to distribute the vapor into the local atmosphere, rather than being inhaled by a user. In an embodiment, vapor exiting the outlet 124 may be at least one of aromatic, medicinal, recreational, and/or wellness related.

In another embodiment, the electronic vaporizing device 100 may comprise a piezoelectric dispersing element 144. In some embodiments, the piezoelectric dispersing element 144 may be charged by a battery, and may be driven by a processor on a circuit board. The circuit board may be produced using a polyimide such as Kapton®, or other suitable material. The piezoelectric dispersing element 144 may comprise a thin metal disc which causes dispersion of the fluid fed into the dispersing element via the wick or other soaked piece of organic material through vibration. Once in contact with the piezoelectric dispersing element 144, the vaporizable material (e.g., fluid) may be vaporized (e.g., turned into vapor or mist) and the vapor may be dispersed via a system pump and/or a sucking action of the user. In some embodiments, the piezoelectric dispersing element 144 may cause dispersion of the vaporizable material by producing ultrasonic vibrations. An electric field applied to a piezoelectric material within the piezoelectric dispersing element 144 may cause ultrasonic expansion and contraction of the piezoelectric material, resulting in ultrasonic vibrations to the disc. The ultrasonic vibrations may cause the vaporizable material to disperse, thus forming a vapor or mist from the vaporizable material.

In some embodiments, the connection between the power supply 120 and the piezoelectric dispersing element 144 may be facilitated using one or more conductive coils. The conductive coils may provide an ultrasonic power input to the piezoelectric dispersing element 144. For example, the signal carried by the coil may have a frequency of approximately 107.8 kHz. In some embodiments, the piezoelectric dispersing element 144 may comprise a piezoelectric dispersing element that may receive the ultrasonic signal transmitted from the power supply through the coils, and may cause cold vaporization (or atomization) of the vaporizable liquid by producing ultrasonic vibrations. An ultrasonic electric field applied to a piezoelectric material within the piezoelectric element causes ultrasonic expansion and contraction of the piezoelectric material, resulting in ultrasonic vibrations according to the frequency of the signal. The vaporizable liquid may be vibrated by the ultrasonic energy produced by the piezoelectric dispersing element 144, thus causing dispersal and/or atomization of the liquid. In an embodiment, the electronic vaporizing device 100 may be configured to permit a user to select between using a heating element of the vaporizer 108 or the piezoelectric dispersing element 144. In another embodiment, the electronic vaporizing device 100 may be configured to permit a user to utilize both a heating element of the vaporizer 108 and the piezoelectric dispersing element 144. The piezoelectric dispersing element 144 is one embodiment of a cold vapor misting distribution component. In other embodiments, the piezoelectric dispersing element 144 may be a cold vapor misting distribution component, such as an atomizer or sprayer that is configured to atomize, pressurize, mist, and/or disperse a vaporizable, atomizable, or mistable material without heat.

In an embodiment, the electronic vaporizing device 100 may comprise a heating casing 126. The heating casing 126 may enclose one or more of the containers 110, the vaporizer 108, and/or the outlet 114. In a further embodiment, the heating casing 126 may enclose one or more components that make up the containers 110, the vaporizer 108, and/or the outlet 114. The heating casing 126 may be made of ceramic, metal, and/or porcelain. The heating casing 126 may have varying thickness. In an embodiment, the heating casing 126 may be coupled to the power supply 120 to receive power to heat the heating casing 126. In another embodiment, the heating casing 126 may be coupled to the vaporizer 108 to heat the heating casing 126. In another embodiment, the heating casing 126 may serve as an insulator.

In an embodiment, the electronic vaporizing device 100 may comprise a filtration element 128. The filtration element 128 may be configured to remove (e.g., filter, purify, etc.) contaminants from air entering the electronic vaporizing device 100. The filtration element 128 may optionally comprise a fan 130 to assist in delivering air to the filtration element 128. The electronic vaporizing device 100 may be configured to intake air into the filtration element 128, filter the air, and pass the filtered air to the vaporizer 108 for use in vaporizing the one or more vaporizable or non-vaporizable materials. In another embodiment, the electronic vaporizing device 100 may be configured to intake air into the filtration element 128, filter the air, and bypass the vaporizer 108 by passing the filtered air directly to the outlet 114 for inhalation by a user.

In an embodiment, the filtration element 128 may comprise cotton, polymer, wool, satin, meta materials, and the like. The filtration element 128 may comprise a filter material that at least one airborne particle and/or undesired gas by a mechanical mechanism, an electrical mechanism, and/or a chemical mechanism. The filter material may comprise one or more pieces of a filter fabric that may filter out one or more airborne particles and/or gasses. The filter fabric may be a woven and/or non-woven material. The filter fabric may be made from natural fibers (e.g., cotton, wool, etc.) and/or from synthetic fibers (e.g., polyester, nylon, polypropylene, etc.). The thickness of the filter fabric may be varied depending on the desired filter efficiencies and/or the region of the apparel where the filter fabric is to be used. The filter fabric may be designed to filter airborne particles and/or gasses by mechanical mechanisms (e.g., weave density), by electrical mechanisms (e.g., charged fibers, charged metals, etc.), and/or by chemical mechanisms (e.g., absorptive charcoal particles, adsorptive materials, etc.). In as embodiment, the filter material may comprise electrically charged fibers such as, but not limited to, Filtrete® by 3M. In another embodiment, the filter material may comprise a high-density material similar to material used for medical masks which are used by medical personnel in doctors' offices, hospitals, and the like. In an embodiment, the filter material may be treated with an anti-bacterial solution and/or otherwise made from anti-bacterial materials. In another embodiment, the filtration element 128 may comprise electrostatic plates, ultraviolet light, a HEPA filter, combinations thereof, and the like.

In an embodiment, the electronic vaporizing device 100 may comprise a cooling element 132. The cooling element 132 may be configured to cool vapor exiting the vaporizer 108 prior to passing through the outlet 114. The cooling element 132 may cool vapor by utilizing air or space within the electronic vaporizing device 100. The air used by the cooling element 132 may be either static (existing in the electronic vaporizing device 100) or drawn into an intake and through the cooling element 132 and the electronic vaporizing device 100. The intake may comprise various pumping, pressure, fan, or other intake systems for drawing air into the cooling element 132. In an embodiment, the cooling element 132 may reside separately or may be integrated the vaporizer 108. The cooling element 132 may be a single cooled electronic element within a tube or space and/or the cooling element 132 may be configured as a series of coils or as a grid like structure. The materials for the cooling element 132 may be metal, liquid, polymer, natural substance, synthetic substance, air, or any combination thereof. The cooling element 132 may be powered by the power supply 120, by a separate battery (not shown), or other power source (not shown) including the use of excess heat energy created by the vaporizer 108 being converted to energy used for cooling by a small turbine or pressure system to convert the energy. Heat differentials between the vaporizer 108 and the cooling element 132 may also be converted to energy utilizing commonly known geothermal energy principles.

In an embodiment, the electronic vaporizing device 100 may comprise a magnetic element 134. For example, the magnetic element 134 may comprise an electromagnet, a ceramic magnet, a ferrite magnet, rare earth magnet, and/or the like. The magnetic element 134 may be configured to apply a magnetic field to air as it is brought into the electronic vaporizing device 100, in the vaporizer 108, and/or as vapor exits the outlet 114.

The input/output device 112 may be used to select whether vapor exiting the outlet 114 should be cooled or not cooled, heated or not heated, and/or magnetized or not magnetized. For example, a user may use the input/output device 112 to selectively cool vapor at times and not cool vapor at other times. The user may use the input/output device 112 to selectively heat vapor at times and not heat vapor at other times. The user may use the input/output device 112 to selectively magnetize vapor at times and not magnetize vapor at other times. The user may further use the input/output device 112 to select a desired smoothness, temperature, and/or range of temperatures. The user may adjust the temperature of the vapor by selecting or clicking on a clickable setting on a part of the electronic vaporizing device 100. The user may use, for example, a graphical user interface (GUI) or a mechanical input enabled by clicking a rotational mechanism at either end of the electronic vaporizing device 100.

In an embodiment, cooling control may be set within the electronic vaporizing device 100 settings via the processor 102 and system software (e.g., dynamic linked libraries). The memory 104 may store settings. Suggestions and remote settings may be communicated to and/or from the electronic vaporizing device 100 via the input/output device 112 and/or the network access device 106. Cooling of the vapor may be set and calibrated between heating and cooling mechanisms to what is deemed an ideal temperature by the manufacturer of the electronic vaporizing device 100 for the vaporizable material. For example, a temperature may be set such that resultant vapor delivers the coolest feeling to the average user but does not present any health risk to the user by the vapor being too cold, including the potential for rapid expansion of cooled vapor within the lungs and the damaging of tissue by vapor which has been cooled to a temperature which may cause frostbite-like symptoms.

In an embodiment, the electronic vaporizing device 100 may be configured to receive air, smoke, vapor or other material and analyze the contents of the air, smoke, vapor or other material using one or more sensors 136 to at least one of analyze, classify, compare, validate, refute, and/or catalogue the same. A result of the analysis may be, for example, an identification of at least one of medical, recreational, homeopathic, olfactory elements, spices, other cooking ingredients, ingredients analysis from food products, fuel analysis, pharmaceutical analysis, genetic modification testing analysis, dating, fossil and/or relic analysis and the like. The electronic vaporizing device 100 may utilize, for example, mass spectrometry, PH testing, genetic testing, particle and/or cellular testing, sensor based testing and other diagnostic and wellness testing, either via locally available components or by transmitting data to a remote system for analysis.

In an embodiment, a user may create a custom scent by using the electronic vaporizing device 100 to intake air elements, wherein the electronic vaporizing device 100 (or third-party networked device) analyzes the olfactory elements and/or biological elements within the sample. The electronic vaporizing device 100 and then formulates a replica scent within the electronic vaporizing device 100 (or third-party networked device) that may be accessed by the user instantly or at a later date, with the ability to purchase this custom scent from a networked ecommerce portal.

In another embodiment, the one or more sensors 136 may be configured to sense negative environmental conditions (e.g., adverse weather, smoke, fire, chemicals (e.g., such as CO2 or formaldehyde), adverse pollution, and/or disease outbreaks, and the like). The one or more sensors 136 may comprise one or more of, a biochemical/chemical sensor, a thermal sensor, a radiation sensor, a mechanical sensor, an optical sensor, a mechanical sensor, a magnetic sensor, an electrical sensor, combinations thereof and the like. The biochemical/chemical sensor may be configured to detect one or more biochemical/chemicals causing a negative environmental condition such as, but not limited to, smoke, a vapor, a gas, a liquid, a solid, an odor, combinations thereof, and the like. The biochemical/chemical sensor may comprise one or more of a mass spectrometer, a conducting/nonconducting regions sensor, a SAW sensor, a quartz microbalance sensor, a conductive composite sensor, a chemiresistor, a metal oxide gas sensor, an organic gas sensor, a MOSFET, a piezoelectric device, an infrared sensor, a sintered metal oxide sensor, a Pd-gate MOSFET, a metal FET structure, an electrochemical cell, a conducting polymer sensor, a catalytic gas sensor, an organic semiconducting gas sensor, a solid electrolyte gas sensors, a piezoelectric quartz crystal sensor, and/or combinations thereof.

The thermal sensor may be configured to detect temperature, heat, heat flow, entropy, heat capacity, combinations thereof, and the like. Exemplary thermal sensors include, but are not limited to, thermocouples, such as semiconducting thermocouples, noise thermometry, thermoswitches, thermistors, metal thermoresistors, semiconducting thermoresistors, thermodiodes, thermotransistors, calorimeters, thermometers, indicators, and fiber optics.

The radiation sensor may be configured to detect gamma rays, X-rays, ultra-violet rays, visible, infrared, microwaves and radio waves. Exemplary radiation sensors are suitable for use in the present invention that include, but are not limited to, nuclear radiation microsensors, such as scintillation counters and solid state detectors; ultra-violet, visible and near infrared radiation microsensors, such as photoconductive cells; photodiodes; phototransistors; infrared radiation microsensors, such as photoconductive IR sensors; and pyroelectric sensors.

The optical sensor may be configured to detect visible, near infrared, and infrared waves. The mechanical sensor may be configured to detect displacement, velocity, acceleration, force, torque, pressure, mass, flow, acoustic wavelength, and amplitude. Exemplary mechanical sensors are suitable for use in the present invention and include, but are not limited to, displacement microsensors, capacitive and inductive displacement sensors, optical displacement sensors, ultrasonic displacement sensors, pyroelectric, velocity and flow microsensors, transistor flow microsensors, acceleration microsensors, piezoresistive microaccelerometers, force, pressure and strain microsensors, and piezoelectric crystal sensors. The magnetic sensor may be configured to detect magnetic field, flux, magnetic moment, magnetization, and magnetic permeability. The electrical sensor may be configured to detect charge, current, voltage, resistance, conductance, capacitance, inductance, dielectric permittivity, polarization and frequency.

Upon sensing a negative environmental condition, the one or more sensors 136 may provide data to the processor 102 to determine the nature of the negative environmental condition and to generate/transmit one or more alerts based on the negative environmental condition. The one or more alerts may be deployed to the electronic vaporizing device 100 user's wireless device and/or synced accounts. For example, the network device access device 106 may be used to transmit the one or more alerts directly (e.g., via Bluetooth®) to a user's smartphone to provide information to the user. In another embodiment, the network access device 106 may be used to transmit sensed information and/or the one or more alerts to a remote server for use in syncing one or more other devices used by the user (e.g., other vapor devices, other electronic devices (smartphones, tablets, laptops, etc.). In another embodiment, the one or more alerts may be provided to the user of the electronic vaporizing device 100 via vibrations, audio, colors, and the like deployed from the mask, for example through the input/output device 112. For example, the input/output device 112 may comprise a small vibrating motor to alert the user to one or more sensed conditions via tactile sensation. In another example, the input/output device 112 may comprise one or more LED's of various colors to provide visual information to the user. In another example, the input/output device 112 may comprise one or more speakers that may provide audio information to the user. For example, various patterns of beeps, sounds, and/or voice recordings may be utilized to provide the audio information to the user. In another example, the input/output device 112 may comprise an LCD screen/touchscreen that provides a summary and/or detailed information regarding the negative environmental condition and/or the one or more alerts.

In another embodiment, upon sensing a negative environmental condition, the one or more sensors 136 may provide data to the processor 102 to determine the nature of the negative environmental condition and to provide a recommendation for mitigating and/or to actively mitigate the negative environmental condition. Mitigating the negative environmental conditions may comprise, for example, applying a filtration system, a fan, a fire suppression system, engaging a HVAC system, and/or one or more vaporizable and/or non-vaporizable materials. The processor 102 may access a database stored in the memory device 104 to make such a determination or the network device 106 may be used to request information from a server to verify the sensor findings. In an embodiment, the server may provide an analysis service to the electronic vaporizing device 100. For example, the server may analyze data sent by the electronic vaporizing device 100 based on a reading from the one or more sensors 136. The server may determine and transmit one or more recommendations to the electronic vaporizing device 100 to mitigate the sensed negative environmental condition. The electronic vaporizing device 100 may use the one or more recommendations to activate a filtration system, a fan, a fire suppression system engaging a HVAC system, and/or to vaporize one or more vaporizable or non-vaporizable materials to assist in countering effects from the negative environmental condition.

In an embodiment, the electronic vaporizing device 100 may comprise a global positioning system (GPS) unit 118. The GPS unit 118 may detect a current location of the device 100. In some embodiments, a user may request access to one or more services that rely on a current location of the user. For example, the processor 102 may receive location data from the GPS 118, convert it to usable data, and transmit the usable data to the one or more services via the network access device 106. The GPS unit 118 may receive position information from a constellation of satellites operated by the U.S. Department of Defense. Alternately, the GPS unit 118 may be a GLONASS receiver operated by the Russian Federation Ministry of Defense, or any other positioning device capable of providing accurate location information (for example, LORAN, inertial navigation, and the like). The GPS unit 118 may contain additional logic, either software, hardware or both to receive the Wide Area Augmentation System (WAAS) signals, operated by the Federal Aviation Administration, to correct dithering errors and provide the most accurate location possible. Overall accuracy of the positioning equipment subsystem containing WAAS is generally in the two-meter range.

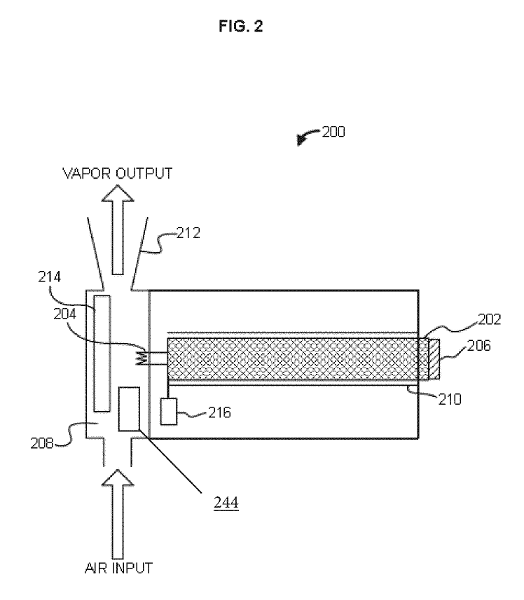

FIG. 2 illustrates one embodiment of an electronic vaporizer 200. The vaporizer 200 may be, for example, an e-cigarette, an e-cigar, an electronic vapor device, a hybrid electronic communication handset coupled/integrated vapor device, a robotic vapor device, a modified vapor device “mod,” a micro-sized electronic vaporizing device, a robotic vapor device, and the like. The vaporizer 200 may be used internally of the electronic vaporizing device 100 or may be a separate device. For example, the vaporizer 200 may be used in place of the vaporizer 108.

The vaporizer 200 may comprise or be coupled to one or more containers 202 containing a vaporizable material, for example a fluid. For example, coupling between the vaporizer 200 and the one or more containers 202 may be via a wick 204, a valve, or by some other coupling/engagement structure. Coupling may operate independently of gravity, such as by capillary action or pressure drop through a valve. The vaporizer 200 may be configured to vaporize the vaporizable material from the one or more containers 202 at controlled rates in response to mechanical input from a component of the electronic vaporizing device 100, and/or in response to control signals from the processor 102 or another component. Vaporizable material (e.g., fluid) may be supplied by one or more replaceable cartridges 206. In an embodiment, the vaporizable material may comprise aromatics and/or aromatic elements. In an embodiment, the aromatic elements may be medicinal, recreational, therapeutic, and/or wellness related. The aromatic element may include, but is not limited to, at least one of lavender or other floral aromatic natural-based liquid compositions, mint, menthol, herbal, extracts, soil or geologic, plant based, name brand perfumes, custom mixed perfume formulated inside the electronic vaporizing device 100 and aromas constructed to replicate the smell of different geographic places, conditions, and/or occurrences. For example, the smell of places may include specific or general sports venues, well known travel destinations, the mix of one's own personal space or home. The smell of conditions may include, for example, the smell of a pet, a baby, a season, a general environment (e.g., a forest), a new car, a sexual nature (e.g., musk, pheromones, etc.). The one or more replaceable cartridges 206 may contain the vaporizable material. If the vaporizable material is liquid, the cartridge may comprise the wick 204 to aid in transporting the liquid to a mixing chamber 208. In the alternative, some other transport mode may be used. Each of the one or more replaceable cartridges 206 may be configured to fit inside and engage removably with a receptacle (such as the container 202 and/or a secondary container) of the electronic vaporizing device 100. In an alternative, or in addition, one or more fluid containers 210 may be fixed in the electronic vaporizing device 100 and configured to be refillable. In an embodiment, one or more materials may be vaporized at a single time by the vaporizer 200. For example, some material may be vaporized and drawn through an exhaust port 212 and/or some material may be vaporized and exhausted via a smoke simulator outlet (not shown).

In operation, a heating element 214 may vaporize or nebulize the vaporizable material in the mixing chamber 208, producing an inhalable vapor/mist that may be expelled via the exhaust port 212. In an embodiment, the heating element 214 may comprise a heater coupled to the wick (or a heated wick) 204 operatively coupled to (for example, in fluid communication with) the mixing chamber 210. The heating element 214 may comprise a nickel-chromium wire or the like, with a temperature sensor (not shown) such as a thermistor or thermocouple. Within definable limits, by controlling power to the wick 204, a rate of vaporization may be independently controlled. Multiplexers 208 and 216 may receive power from a vaporizer power supply 218 and/or from a power supply 120 built into the electronic vaporizing device 100. At a minimum, control may be provided between no power (off state) and one or more powered states. Other control mechanisms may also be suitable.

In another embodiment, the vaporizer 200 may comprise a piezoelectric dispersing element 244. In some embodiments, the piezoelectric dispersing element 244 may be charged by a battery, and may be driven by a processor on a circuit board. The circuit board may be produced using a polyimide such as Kapton®, or other suitable material. The piezoelectric dispersing element 244 may comprise a thin metal disc which causes dispersion of the fluid fed into the dispersing element via the wick or other soaked piece of organic material through vibration. Once in contact with the piezoelectric dispersing element, the vaporizable material (e.g., fluid) may be vaporized (e.g., turned into vapor or mist) and the vapor may be dispersed via a system pump and/or a sucking action of the user. In some embodiments, the piezoelectric dispersing element 244 may cause dispersion of the vaporizable material by producing ultrasonic vibrations. An electric field applied to a piezoelectric material within the piezoelectric element may cause ultrasonic expansion and contraction of the piezoelectric material, resulting in ultrasonic vibrations to the disc. The ultrasonic vibrations may cause the vaporizable material to disperse, thus forming a vapor or mist from the vaporizable material.

In an embodiment, the vaporizer 200 may be configured to permit a user to select between using the heating element 214 or the piezoelectric dispersing element 244. In another embodiment, the vaporizer 200 may be configured to permit a user to utilize both the heating element 214 and the piezoelectric dispersing element 244.

In some embodiments, the connection between a power supply and the piezoelectric dispersing element 244 may be facilitated using one or more conductive coils. The conductive coils may provide an ultrasonic power input to the piezoelectric dispersing element 244. For example, the signal carried by the coil may have a frequency of approximately 107.8 kHz. In some embodiments, the piezoelectric dispersing element 244 may comprise a piezoelectric dispersing element that may receive the ultrasonic signal transmitted from the power supply through the coils, and may cause vaporization of the vaporizable liquid by producing ultrasonic vibrations. An ultrasonic electric field applied to a piezoelectric material within the piezoelectric dispersing element 244 causes ultrasonic expansion and contraction of the piezoelectric material, resulting in ultrasonic vibrations according to the frequency of the signal. The vaporizable liquid may be vibrated by the ultrasonic energy produced by the piezoelectric dispersing element 244, thus causing dispersal and/or atomization of the liquid.

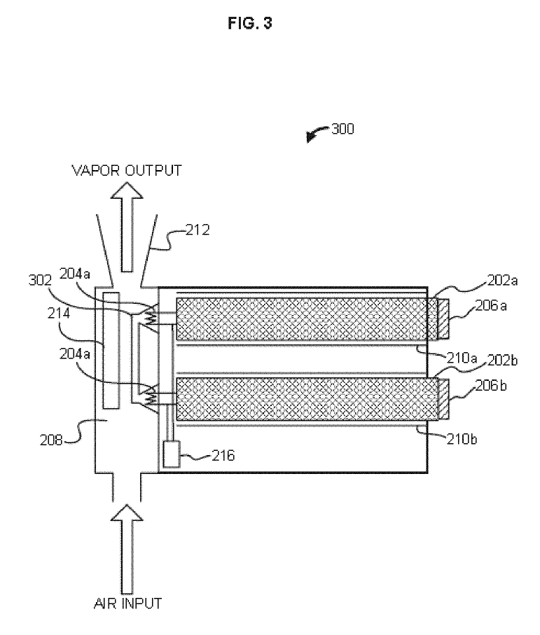

FIG. 3 illustrates one embodiment of a vaporizer 300 that comprises the elements of the vaporizer 200 with two containers 202a and 202b containing a vaporizable material, for example a fluid. In an embodiment, the fluid may be the same fluid in both containers or the fluid may be different in each container. In an embodiment, the fluid may comprise aromatic elements. The aromatic element may include, but is not limited to, at least one of lavender or other floral aromatic natural-based liquid compositions, mint, menthol, herbal soil or geologic, plant based, name brand perfumes, custom mixed perfume formulated inside the electronic vaporizing device 100 and aromas constructed to replicate the smell of different geographic places, conditions, and/or occurrences. For example, the smell of places may include specific or general sports venues, well known travel destinations, the mix of one's own personal space or home. The smell of conditions may include, for example, the smell of a pet, a baby, a season, a general environment (e.g., a forest), a new car, a sexual nature (e.g., musk, pheromones, etc.). Coupling between the vaporizer 200 and the container 202a and the container 202b may be via a wick 204a and a wick 204b, respectively, via a valve, or by some other structure. Coupling may operate independently of gravity, such as by capillary action or pressure drop through a valve. The vaporizer 300 may be configured to mix in varying proportions the fluids contained in the container 202a and the container 202b and vaporize the mixture at controlled rates in response to mechanical input from a component of the electronic vaporizing device 100, and/or in response to control signals from the processor 102 or another component. In an embodiment, a mixing element 302 may be coupled to the container 202a and the container 202b. The mixing element may 302, in response to a control signal from the processor 102, withdraw select quantities of vaporizable material to create a customized mixture of different types of vaporizable material. Vaporizable material (e.g., fluid) may be supplied by one or more replaceable cartridges 206a and 206b. The one or more replaceable cartridges 206a and 206b may contain a vaporizable material. If the vaporizable material is liquid, the cartridge may comprise the wick 204a or 204b to aid in transporting the liquid to a mixing chamber 208. In the alternative, some other transport mode may be used. Each of the one or more replaceable cartridges 206a and 206b may be configured to fit inside and engage removably with a receptacle (such as the container 202a or the container 202b and/or a secondary container) of the electronic vaporizing device 100. In an alternative, or in addition, one or more fluid containers 210a and 210b may be fixed in the electronic vaporizing device 100 and configured to be refillable. In an embodiment, one or more materials may be vaporized at a single time by the vaporizer 300. For example, some material may be vaporized and drawn through an exhaust port 212 and/or some material may be vaporized and exhausted via a smoke simulator outlet (not shown).

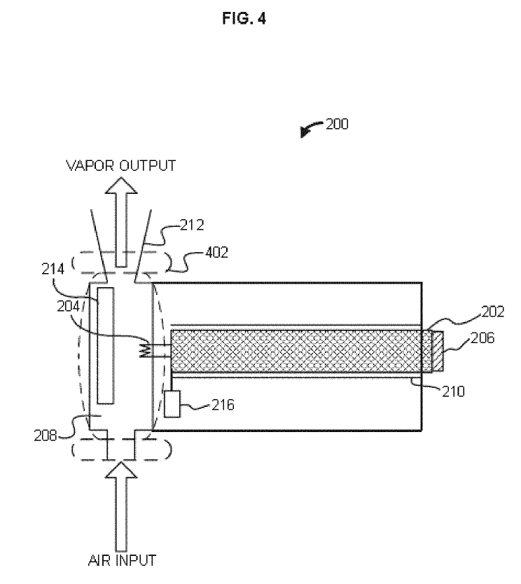

FIG. 4 illustrates one embodiment of a vaporizer 200 that comprises the elements of the vaporizer 200 with a heating casing 402. The heating casing 402 may enclose the heating element 214 or may be adjacent to the heating element 214. The heating casing 402 is illustrated with dashed lines, indicating components contained therein. The heating casing 402 may preferably be made of ceramic, metal, and/or porcelain. The heating casing 402 may have varying thickness. In an embodiment, the heating casing 402 may be coupled to the multiplexer 216 to receive power to heat the heating casing 402. In another embodiment, the heating casing 402 may be coupled to the heating element 214 to heat the heating casing 402. In another embodiment, the heating casing 402 may serve as an insulator.

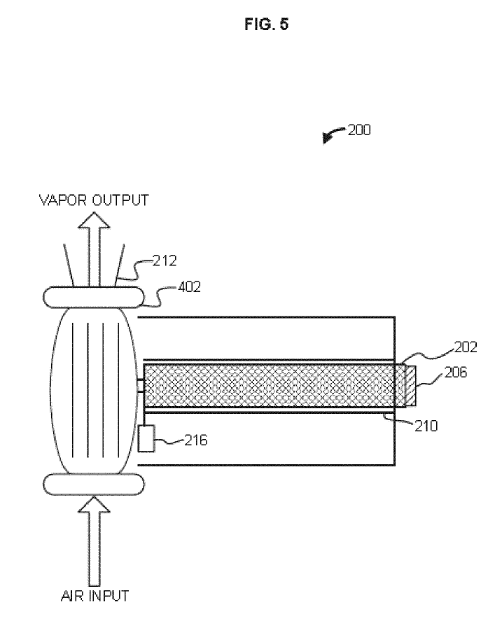

FIG. 5 illustrates one embodiment of the vaporizer 200 of FIG. 4, but illustrates the heating casing 402 with solid lines, indicating components contained therein. Other placements of the heating casing 402 are contemplated. For example, the heating casing 402 may be placed after the heating element 214 and/or the mixing chamber 208.

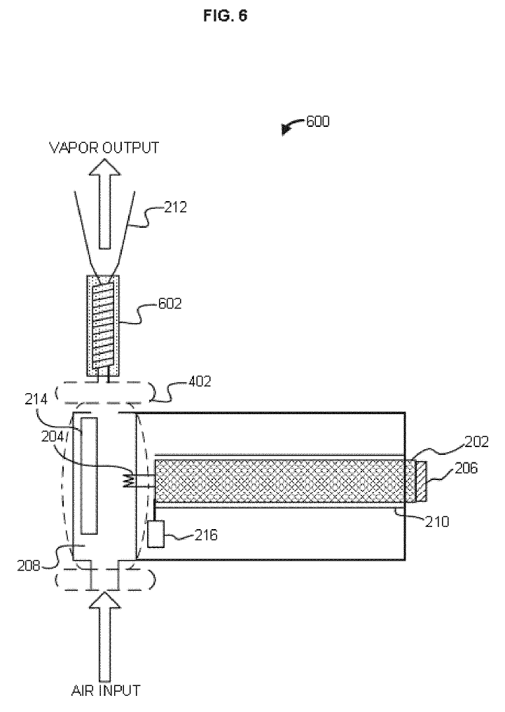

FIG. 6 illustrates one embodiment of a vaporizer 600 that comprises the elements of the vaporizer 200 of FIG. 2 and FIG. 4, with the addition of a cooling element 602. The vaporizer 600 may optionally comprise the heating casing 402. The cooling element 602 may comprise one or more of a powered cooling element, a cooling air system, and/or or a cooling fluid system. The cooling element 602 may be self-powered, co-powered, or directly powered by a battery and/or charging system within the electronic vaporizing device 100 (e.g., the power supply 120). In an embodiment, the cooling element 602 may comprise an electrically connected conductive coil, grating, and/or other design to efficiently distribute cooling to the vaporized and/or non-vaporized air. For example, the cooling element 602 may be configured to cool air as it is brought into the vaporizer 600/mixing chamber 208 and/or to cool vapor after it exits the mixing chamber 208. The cooling element 602 may be deployed such that the cooling element 602 is surrounded by the heated casing 402 and/or the heating element 214. In another embodiment, the heated casing 402 and/or the heating element 214 may be surrounded by the cooling element 602. The cooling element 602 may utilize at least one of cooled air, cooled liquid, and/or cooled matter.

In an embodiment, the cooling element 602 may be a coil of any suitable length and may reside proximate to the inhalation point of the vapor (e.g., the exhaust port 212). The temperature of the air is reduced as it travels through the cooling element 602. In an embodiment, the cooling element 602 may comprise any structure that accomplishes a cooling effect. For example, the cooling element 602 may be replaced with a screen with a mesh or grid-like structure, a conical structure, and/or a series of cooling airlocks, either stationary or opening, in a periscopic/telescopic manner. The cooling element 602 may be any shape and/or may take multiple forms capable of cooling heated air, which passes through its space.

In an embodiment, the cooling element 602 may be any suitable cooling system for use in a vapor device. For example, a fan, a heat sink, a liquid cooling system, a chemical cooling system, combinations thereof, and the like. In an embodiment, the cooling element 602 may comprise a liquid cooling system whereby a fluid (e.g., water, coolant) passes through pipes in the vaporizer 600. As this fluid passes around the cooling element 602, the fluid absorbs heat, cooling the air in the cooling element 602. After the fluid absorbs the heat, the fluid may pass through a heat exchanger which transfers the heat from the fluid to air blowing through the heat exchanger. By way of further example, the cooling element 602 may comprise a chemical cooling system that utilizes an endothermic reaction. An example of an endothermic reaction is dissolving ammonium nitrate in water. Such endothermic process is used in instant cold packs. These cold packs have a strong outer plastic layer that holds a bag of water and a chemical, or mixture of chemicals, that result in an endothermic reaction when dissolved in water. When the cold pack is squeezed, the inner bag of water breaks and the water mixes with the chemicals. The cold pack starts to cool as soon as the inner bag is broken, and stays cold for over an hour. Many instant cold packs contain ammonium nitrate. When ammonium nitrate is dissolved in water, it splits into positive ammonium ions and negative nitrate ions. In the process of dissolving, the water molecules contribute energy, and as a result, the water cools down. Thus, the vaporizer 600 may comprise a chamber for receiving the cooling element 602 in the form of a “cold pack.” The cold pack may be activated prior to insertion into the vaporizer 600 or may be activated after insertion through use of a button/switch and the like to mechanically activate the cold pack inside the vaporizer 600.

In an embodiment, the cooling element 602 may be selectively moved within the vaporizer 600 to control the temperature of the air mixing with vapor. For example, the cooling element 602 may be moved closer to the exhaust port 212 or further from the exhaust port 212 to regulate temperature. In another embodiment, insulation may be incorporated as needed to maintain the integrity of heating and cooling, as well as absorbing any unwanted condensation due to internal or external conditions, or a combination thereof. The insulation may also be selectively moved within the vaporizer 600 to control the temperature of the air mixing with vapor. For example, the insulation may be moved to cover a portion, none, or all of the cooling element 602 to regulate temperature.

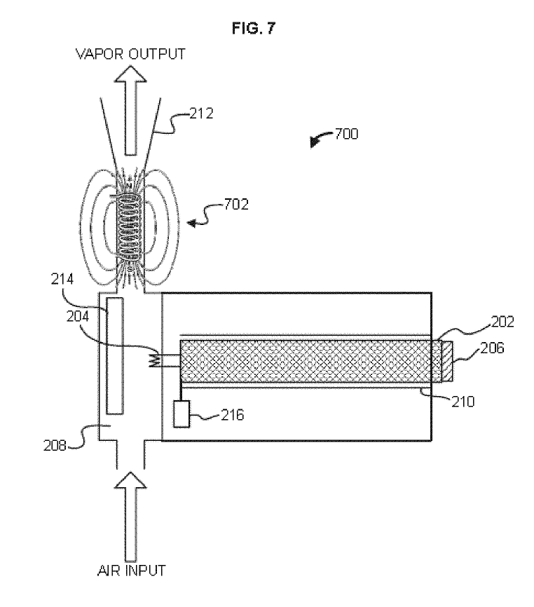

FIG. 7 illustrates one embodiment of a vaporizer 700 that comprises elements in common with the vaporizer 200. The vaporizer 700 may optionally comprise a heating casing (not shown) and/or cooling element (not shown) as discussed above. The vaporizer 700 may comprise a magnetic element 702. The magnetic element 702 may apply a magnetic field to vapor after exiting the mixing chamber 208. The magnetic field may cause positively and negatively charged particles in the vapor to curve in opposite directions, according to the Lorentz force law with two particles of opposite charge. The magnetic field may be created by at least one of an electric current generating a charge or a pre-charged magnetic material deployed within the electronic vaporizing device 100. In an embodiment, the magnetic element 702 may be built into the mixing chamber 208, the cooling element 602, the heating casing 402, or may be a separate magnetic element 702.

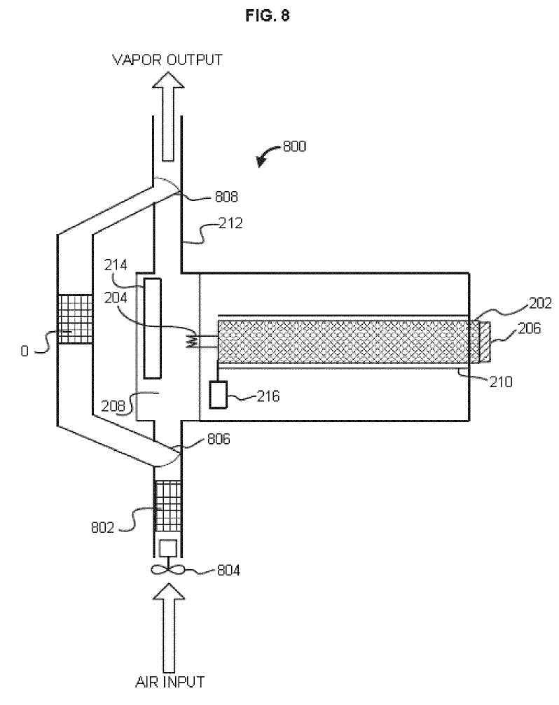

FIG. 8 illustrates one embodiment of a vaporizer 800 that comprises elements in common with the vaporizer 200. In an embodiment, the vaporizer 800 may comprise a filtration element 802. The filtration element 802 may be configured to remove (e.g., filter, purify, etc.) contaminants from air entering the vaporizer 800. The filtration element 802 may optionally comprise a fan 804 to assist in delivering air to the filtration element 802. The vaporizer 800 may be configured to intake air into the filtration element 802, filter the air, and pass the filtered air to the mixing chamber 208 for use in vaporizing the one or more vaporizable or non-vaporizable materials. In another embodiment, the vaporizer 800 may be configured to intake air into the filtration element 802, filter the air, and bypass the mixing chamber 208 by engaging a door 806 and a door 808 to pass the filtered air directly to the exhaust port 212 for inhalation by a user. In an embodiment, filtered air that bypasses the mixing chamber 208 by engaging the door 806 and the door 808 may pass through a second filtration element 810 to further remove (e.g., filter, purify, etc.) contaminants from air entering the vaporizer 800. In an embodiment, the vaporizer 800 may be configured to deploy and/or mix a proper/safe amount of oxygen which may be delivered either via the one or more replaceable cartridges 206 or via air pumped into a mask from external air and filtered through the filtration element 802 and/or the filtration element 810.

In an embodiment, the filtration element 802 and/or the filtration element 810 may comprise cotton, polymer, wool, satin, meta materials and the like. The filtration element 802 and/or the filtration element 810 may comprise a filter material that at least one airborne particle and/or undesired gas by a mechanical mechanism, an electrical mechanism, and/or a chemical mechanism. The filter material may comprise one or more pieces of, a filter fabric that may filter out one or more airborne particles and/or gasses. The filter fabric may be a woven and/or non-woven material. The filter fabric may be made from natural fibers (e.g., cotton, wool, etc.) and/or from synthetic fibers (e.g., polyester, nylon, polypropylene, etc.). The thickness of the filter fabric may be varied depending on the desired filter efficiencies and/or the region of the apparel where the filter fabric is to be used. The filter fabric may be designed to filter airborne particles and/or gasses by mechanical mechanisms (e.g., weave density), by electrical mechanisms (e.g., charged fibers, charged metals, etc.), and/or by chemical mechanisms (e.g., absorptive charcoal particles, adsorptive materials, etc.). In as embodiment, the filter material may comprise electrically charged fibers such as, but not limited to, Filtrete® by 3M. In another embodiment, the filter material may comprise a high-density material similar to material used for medical masks which are used by medical personnel in doctors' offices, hospitals, and the like. In an embodiment, the filter material may be treated with an anti-bacterial solution and/or otherwise made from anti-bacterial materials. In another embodiment, the filtration element 802 and/or the filtration element 810 may comprise electrostatic plates, ultraviolet light, a HEPA filter, combinations thereof, and the like.

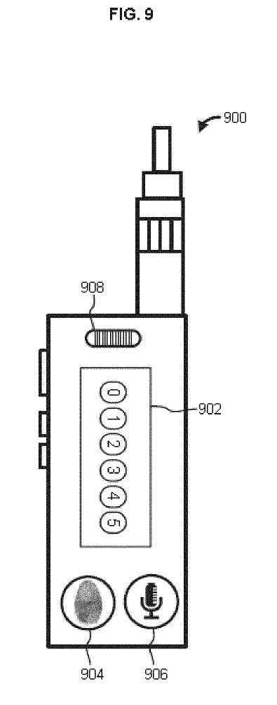

FIG. 9 illustrates one embodiment of a vapor device 900. The exemplary vapor device 900 may comprise the electronic vaporizing device 100 and/or any of the vaporizers 200, 600, 700, 800 disclosed herein. The vapor device 900 illustrates a display 902. The display 902 may be a touchscreen. The display 902 may be configured to enable a user to control any and/or all functionality of the vapor device 900. For example, a user may utilize the display 902 to enter a pass code to lock and/or unlock the vapor device 900. The vapor device 900 may comprise a biometric interface 904. For example, the biometric interface 904 may comprise a fingerprint scanner, an eye scanner, a facial scanner, and the like. The biometric interface 904 may be configured to enable a user to control any and/or all functionality of the vapor device 900. The vapor device 900 may comprise an audio interface 906. The audio interface 906 may comprise a button that, when engaged, enables a microphone 908. The microphone 908 may receive audio signals and provide the audio signals to a processor for interpretation into one or more commands to control one or more functions of the vapor device 900.

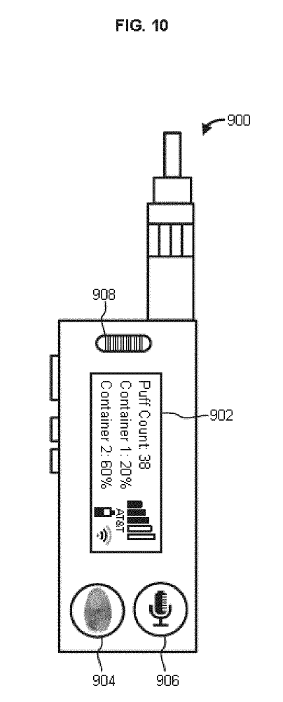

FIG. 10 illustrates one embodiment of exemplary information that may be provided to a user via the display 902 of the vapor device 900. The display 902 may provide information to a power remaining in one or more power supplied, signal strength, combinations thereof, and the like. The display 902 is preferably digital, but may be analog.

In view of the exemplary systems described herein, methodologies that may be implemented in accordance with the disclosed subject matter have been described with reference to several flow diagrams. While for purposes of simplicity of explanation, the methodologies are shown and described as a series of blocks, it is to be understood and appreciated that the claimed subject matter is not limited by the order of the blocks, as some blocks may occur in different orders and/or concurrently with other blocks from what is depicted and described herein. Moreover, not all illustrated blocks may be required to implement the methodologies described herein. Additionally, it should be further appreciated that the methodologies disclosed herein are capable of being stored on an article of manufacture to facilitate transporting and transferring such methodologies to computers.

Those of ordinary skill in the relevant art would further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

As used in this application, the terms “component,” “module,” “system,” and the like are intended to refer to a computer-related entity, either hardware, a combination of hardware and software, software, or software in execution. For example, a component may be, but is not limited to being, a process running on a processor, a processor, an object, an executable, a thread of execution, a program, and/or a computer. By way of illustration, both an application running on a server and the server may be a component. One or more components may reside within a process and/or thread of execution and a component may be localized on one computer and/or distributed between two or more computers.

As used herein, a “vapor” includes mixtures of a carrier gas or gaseous mixture (for example, air) with any one or more of a dissolved gas, suspended solid particles, or suspended liquid droplets, wherein a substantial fraction of the particles or droplets if present are characterized by an average diameter of not greater than three microns. As used herein, an “aerosol” has the same meaning as “vapor,” except for requiring the presence of at least one of particles or droplets. A substantial fraction means 10% or greater; however, it should be appreciated that higher fractions of small (<3 micron) particles or droplets may be desirable, up to and including 100%. It should further be appreciated that, to simulate smoke, average particle or droplet size may be less than three microns, for example, may be less than one micron with particles or droplets distributed in the range of 0.01 to 1 micron. A vaporizer may include any device or assembly that produces a vapor or aerosol from a carrier gas or gaseous mixture and at least one vaporizable material. An aerosolizer is a species of vaporizer, and as such is included in the meaning of vaporizer as used herein, except where specifically disclaimed.

Various embodiments presented in terms of systems may comprise a number of components, modules, and the like. It is to be understood and appreciated that the various systems may include additional components, modules, etc. and/or may not include all of the components, modules, etc. discussed in connection with the figures. A combination of these approaches may also be used.

In addition, the various illustrative logical blocks, modules, and circuits described in connection with certain embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, system-on-a-chip, or state machine. A processor may also be implemented as a combination of computing devices, e.g., a combination of a DSP and a microprocessor, a plurality of microprocessors, one or more microprocessors in conjunction with a DSP core, or any other such configuration.

Operational embodiments disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, a DVD disk, or any other form of storage medium known in the art. An exemplary storage medium is coupled to the processor such the processor may read information from, and write information to, the storage medium. In the alternative, the storage medium may be integral to the processor. The processor and the storage medium may reside in an ASIC or may reside as discrete components in another device.

Furthermore, the one or more versions may be implemented as a method, apparatus, or article of manufacture using standard programming and/or engineering techniques to produce software, firmware, hardware, or any combination thereof to control a computer to implement the disclosed embodiments. Non-transitory computer readable media may include but are not limited to magnetic storage devices (e.g., hard disk, floppy disk, magnetic strips), optical disks (e.g., compact disk (CD), digital versatile disk (DVD)), smart cards, and flash memory devices (e.g., card, stick). Those skilled in the art will recognize many modifications may be made to this configuration without departing from the scope of the disclosed embodiments.

The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those of ordinary skill in the art that various modifications and variations may be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A natural-based liquid composition for use in an electronic vaporizing device comprising:
about 70 weight percent to about 99 weight percent of water; and
about 0.01 weight percent to about 10 weight percent of nicotine derived from at least one non-tobacco nicotine source;
wherein the natural-based liquid composition is substantially free of at least one of propylene glycol and vegetable glycerin.

2. The natural-based liquid composition of claim 1, wherein the at least one non-tobacco nicotine source is selected from the Solanaceae plant family.

3. The natural-based liquid composition of claim 1, wherein the at least one non-tobacco nicotine source is selected from the group of non-tobacco nicotine sources consisting of at least one of duboisia hopwoodii, eggplant, tomatoes, potatoes, green peppers, and combinations thereof.

4. The natural-based liquid composition of claim 3, wherein the at least one non-tobacco nicotine source is duboisia hopwoodii.

5. The natural-based liquid composition of claim 1, further comprising about 0.01 weight percent to about 10 weight percent of nornicotine derived from at least one non-tobacco nornicotine source.

6. The natural-based liquid composition of claim 1, further comprising about 1 weight percent to about 30 weight percent of at least one flavoring agent.

7. The natural-based liquid composition of claim 6, wherein the at least one flavoring agent is selected from the group of flavoring agents consisting of at least one of a fruit, a berry, a spice, an herb, a savory flavor, a spicy flavor, a sweet flavor, a plant-based flavor, and combinations thereof.

8. The natural-based liquid composition of claim 1, further comprising about 0.01 weight percent to about 20 weight percent of at least one natural emulsifier.

9. The natural-based liquid composition of claim 8, wherein the at least one natural emulsifier is selected from the group of natural emulsifiers consisting of at least one of a sunflower, a lecithin, a natural starch, and combinations thereof.

10. The natural-based liquid composition of claim 1, further comprising about 0.01 weight percent to about 10 weight percent of at least one supplementary component selected from the group of supplementary components consisting of at least one of a medicinal agent, a wellness agent, a recreational use agent, and combinations thereof.

11. The natural-based liquid composition of claim 10, wherein the supplementary component is a medicinal agent that is selected from the group of medicinal agents consisting of at least one of a diabetes medication, a respiratory medication, a sexual dysfunction medication, a cannabis-based medication, and combinations thereof.

12. The natural-based liquid composition of claim 10, wherein the supplementary component is a wellness agent that is selected from the group wellness agents consisting of at least one of a chamomile, Echinacea, a homeopathic remedy, a vitamin supplement, and combinations thereof.

13. The natural-based liquid composition of claim 10, wherein the supplementary component is a recreational use agent that is selected from the group of recreational use agents consisting of at least one of caffeine, a cannabis-based material, taurine, salvia, kratum, kava, and combinations thereof.

14. The natural-based liquid composition of claim 1, wherein the composition is substantially free of both propylene glycol and vegetable glycerin.

15. A natural-based liquid composition for use in an electronic vaporizing device comprising:
about 70 weight percent to about 99 weight percent of water;
about 0.01 weight percent to about 10 weight percent of nicotine derived from at least one non-tobacco nicotine source; and
about 0.01 weight percent to about 20 weight percent of at least one natural emulsifier;
wherein the natural-based liquid composition is substantially free of both propylene glycol and vegetable glycerin.

16. A method for producing a natural-based liquid composition comprising the steps:
combining about 70 weight percent to about 99 weight percent of water with about 0.01 weight percent to about 10 weight percent of nicotine derived from at least one non-tobacco nicotine source to form a liquid solution;
adding about 0.01 weight percent to about 20 weight percent of at least one natural emulsifier to the liquid solution; and
mixing the liquid composition containing the at least one natural emulsifier to form a substantially homogeneous liquid composition.

17. The method of claim 16, wherein the at least one non-tobacco nicotine source is selected from the Solanaceae plant family.

18. The method of claim 16, wherein the at least one non-tobacco nicotine source is selected from the group of non-tobacco nicotine sources consisting of at least one of duboisia hopwoodii, eggplant, tomatoes, potatoes, green peppers, and combinations thereof.

19. The method of claim 16, wherein the at least one natural emulsifier is selected from the group of natural emulsifiers consisting of at least one of a sunflower, a lecithin, a natural starch, and combinations thereof.

20. The method of claim 16, further comprising the steps:
adding about 0.01 weight percent to about 10 weight percent of at least one supplementary component to the liquid solution, wherein the at least one supplementary component is selected from the group of supplementary components consisting of at least one of a flavoring agent, a medicinal agent, a wellness agent, a recreational use agent, nornicotine, and combinations thereof.
